(19) United States
(12) Patent Application Publication
(10) Pub. No.: US 2024/0309023 A1
(43) Pub. Date: Sep. 19, 2024

(54) MOLECULAR SORBENT COMPOSITIONS AND METHODS OF USE THEREOF

(71) Applicant: THE BOARD OF REGENTS OF THE UNIVERSITY OF TEXAS SYSTEM, Austin, TX (US)

(72) Inventors: Rasika DIAS, Arlington, TX (US); Anurag Noonikara POYIL, Arlington, TX (US); Banglin CHEN, San Antonio, TX (US); Hui CUI, San Antonio, TX (US)

(21) Appl. No.: 18/577,066

(22) PCT Filed: Jul. 6, 2022
(86) PCT No.: PCT/US2022/073461

§ 371 Date: Jan. 5, 2024

Related U.S. Application Data
(60) Provisional application No. 63/218,569, filed on Jul. 6, 2021.

(51) Int. Cl. C07F 5/05; B01J 20/22; C07C 7/11; C07C 7/12

(52) U.S. Cl. CPC C07F 5/05; B01J 20/223; C07C 7/11; C07C 7/12; B01J 2220/58

(57) ABSTRACT

Provided herein is a molecular sorbent based on copper that provides high-purity separation, such as separation of ethylene from ethylene-ethane mixtures and propylene from propylene-propane mixtures. Further provided herein are compositions thereof, methods of manufacturing these, and methods of use thereof.

This application claims the benefit of priority to U.S. Provisional Application No. 63/218,569, filed on Jul. 6, 2021, the entire content of which are hereby incorporated by reference.

This invention was made with government support under Grant No. CHE-1954456 awarded by the National Science Foundation (NSF). The government has certain rights in the invention.

### BACKGROUND

### I. Field

The present disclosure relates generally to the fields of chemistry and materials science. More particularly, it concerns molecular sorbents, methods of preparation thereof, compositions thereof and methods of use thereof, including separating gas molecules, such as ethylene, ethane, propylene, and propane.

### II. Description of Related Art

Ethylene, the largest-volume organic product of the chemical industry, is produced mainly by the steam-cracking of petroleum feedstocks. This process results in a mixture of gaseous hydrocarbons that includes ethylene (C₂H₄) and ethane (C₂H₆). Industrial purification of ethylene from ethane is achieved by energy consuming, cryogenic distillation, which requires distillation columns with over 100 trays operating at temperatures around −25° C. and pressures >2000 kPa due to similarities in volatility (Sholl David and Lively Ryan et al., 2016; Chu et al., 2016). There have been extensive research endeavours to pursue materials for the cost-and energy-efficient separation of ethylene purification over the past half-decade but without significant progress. The emergence of porous metal-organic framework materials (MOFs) has primarily provided a promising option for this industrial gas separations given the fact that the pores of MOFs can be readily tuned for the sieving separations and functionalized for the specific recognition of one gas molecule over another one (Li et al., 2019; Lin et al., 2020). In fact, the inventors and other groups have successfully targeted several high-performance MOF materials for ethylene purification over the past several years (Bloch et al., 2012; He et al., 2012; Yang et al., 2015; Li et al., 2014; Aguado et al., 2012; Bereciartua et al., 2017; Li et al., 2018; Lin et al., 2018). Previous studies on the ultramicroporous metal-organic framework [Ca(C₄O₄)(H₂O)] (UTSA-280) have shown this MOF to be selective for ethylene separation (Lin et al., Nat. Mater., 17(12):1128, 2018 and WO 2019/183635, both of which are incorporated by reference herein in their entireties).

Ethylene is also a plant hormone with many roles in growth and development including seed germination, fruit ripening, and senescence (Binder, 2020). Studies on ethylene receptor ETR1 indicate that copper is the cofactor in this protein, which binds ethylene quite tightly (K_{d}=2.4×10⁻⁹ M and a half-life for ethylene dissociation of 12.5 h) (Rodriguez et al., 1999; Schaller et al., 1995; Schott-Verdugo et al., 2019). Poly(pyrazolyl)borates, often referred to as scorpionates (Trofimenko et al., 1993), are a useful family of ligands for many applications including modelling ethylene receptor site of plants and obtaining isolable copper-ethylene complexes such as the tris(pyrazolyl)borate [(CH₃)₂Tp]Cu(C₂H₄)(1) (Thompson et al., 1983; Dias et al., 2008). In contrast to most copper(I) ethylene complexes which are quite air sensitive and labile (Munakata et al., 1986; Straub et al., 1999), the fluorinated analogue, [(CF₃)₂Tp]Cu(C₂H₄) (2) is a thermally stable solid and quite resistant to O₂, and the loss of coordinated ethylene (Dias et al., 2002). Considering the current interest in non-porous materials that can facilitate ethylene-ethane separation without the need for cryogenic distillation (Parasar et al., 2020; Jayaratna et al., 2018; Cowan et al., 2015), it would be desirable to develop molecules that bind ethylene.

Given the usefulness of materials that can effectively separate industrial feedstocks, such as ethylene from ethane, or propylene from propane, materials that can achieve these separations are of great importance, including methods and processes to fabricate these materials.

### SUMMARY

In some aspects, the present disclosure provides new molecular sorbents, compositions thereof, methods of manufacturing these, and methods of use thereof. In one aspect, the present disclosure provides a compound of the formula:

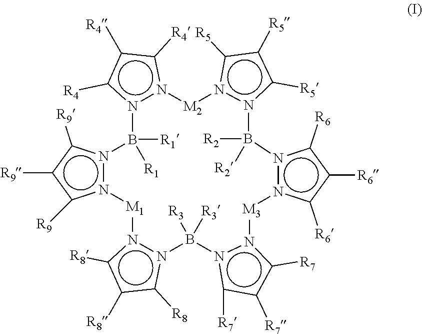

wherein:
R₁, R₁′, R₂, R₂′, R₃, and R₃′ are each independently selected from hydrogen, alkyl_{(C≤6)}, or substituted alkyl_{(C≤6)};
M₁, M₂, or M₃ are each independently selected from a monovalent metal ion; and
R₄, R₄′, R₅, R₅′, R₆, R₆′, R₇, R₇′, R₈, R₈′, R₉, and R₉′ are each independently alkyl_{(C≤12)}, substituted alkyl_{(C≤12)}, aryl_{(C≤12)}, or substituted aryl_{(C≤12)}; and
R₄″, R₅″, R₆″, R₇″, R₈″, and R₉″ is hydrogen, amino, cyano, halo, hydroxy, nitro, alkyl_{(C≤12)}, aryl_{(C≤12)}, acyl_{(C≤12)}, alkoxy_{(C≤12)}, alkylamino_{(C≤12)}, dialkylamino_{(C≤12)}, or a substituted version thereof.

In some embodiments, the compound is further defined as:

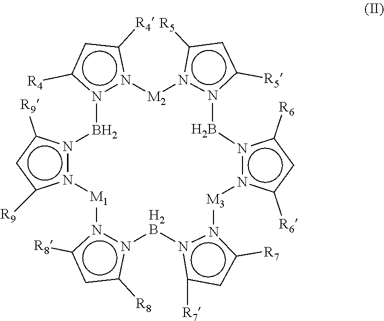

wherein:
M₁, M₂, or M₃ are each independently selected from a monovalent metal ion; and
R₄, R₄′, R₅, R₅′, R₆, R₆′, R₇, R₇′, R₈, R₈′, R₉, and R₉′ are each independently alkyl_{(C≤12)}, substituted alkyl_{(C≤12)}, aryl_{(C≤12)}, or substituted aryl_{(C≤12)}.

In some embodiments, the compound is further defined as:

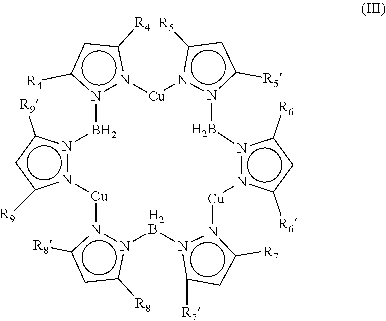

wherein:
R₄, R₄′, R₅, R₅′, R₆, R₆′, R₇, R₇′, R₈, R₈′, R₉, and R₉′ are each independently alkyl_{(C≤12)}, substituted alkyl_{C≤12)}, aryl_{(C≤12)}, or substituted aryl_{(C≤12)}.

In some embodiments, R₄ is substituted alkyl_{(C≤12)}. In some embodiments, R₄ is substituted alkyl_{(C≤6)}. In some embodiments, R₄ is haloalkyl_{(C≤6)}. In some embodiments, R₄ is trifluoromethyl.

In some embodiments, R₄′ is substituted alkyl_{(C≤12)}. In some embodiments, R₄′ is substituted alkyl_{(C≤6)}. In some embodiments, R₄′ is haloalkyl_{(C≤6)}. In some embodiments, R₄′ is trifluoromethyl.

In some embodiments, R₅ is substituted alkyl_{(C≤12)}. In some embodiments, R₅ is substituted alkyl_{(C≤6)}. In some embodiments, R₅ is haloalkyl_{(C≤6)}. R₅ is trifluoromethyl.

In some embodiments, R₅′ is substituted alkyl_{(C≤12)}. In some embodiments, R₅′ is substituted alkyl_{(C≤6)}. In some embodiments, R₅′ is haloalkyl_{(C≤6)}. In some embodiments, R₅′ is trifluoromethyl.

In some embodiments, R₆ is substituted alkyl_{(C≤12)}. In some embodiments, R₆ is substituted alkyl_{(C≤6)}. In some embodiments, R₆ is substituted alkyl_{(C≤6)}. In some embodiments, R₆ is trifluoromethyl.

In some embodiments, R₆′ is substituted alkyl_{(C≤12)}. In some embodiments, R₆′ is substituted alkyl_{(C≤6)}. In some embodiments, R₆′ is substituted alkyl_{(C≤6)}. In some embodiments, R₆′ is trifluoromethyl.

In some embodiments, R₇ is substituted alkyl_{(C≤12)}. In some embodiments, R₇ is substituted alkyl_{(C≤6)}. In some embodiments, R₇ is substituted alkyl_{(C≤6)}. In some embodiments, R₇ is trifluoromethyl. In some embodiments, R₇′ is substituted alkyl_{(C≤12)}.

In some embodiments, R₇′ is substituted alkyl_{(C≤6)}. In some embodiments, R₇′ is substituted alkyl_{(C≤6)}. In some embodiments, R₇′ is trifluoromethyl.

In some embodiments, R₈ is substituted alkyl_{(C≤12)}. In some embodiments, R₈ is substituted alkyl_{(C≤6)}. In some embodiments, R₈ is substituted alkyl_{(C≤6)}. In some embodiments, R₈ is trifluoromethyl.

In some embodiments, R₈′ is substituted alkyl_{(C≤12)}. In some embodiments, R₈′ is substituted alkyl_{(C≤6)}. In some embodiments, R₈′ is substituted alkyl_{(C≤6)}. In some embodiments, R₈′ is trifluoromethyl.

In some embodiments, R₉ is substituted alkyl_{(C≤12)}. In some embodiments, R₉ is substituted alkyl_{(C≤6)}. In some embodiments, R₉ is substituted alkyl_{(C≤6)}. In some embodiments, R₉ is trifluoromethyl.

In some embodiments, R₉′ is substituted alkyl_{(C≤12)}. In some embodiments, R₉′ is substituted alkyl_{(C≤6)}. In some embodiments, R₉′ is substituted alkyl_{(C≤6)}. In some embodiments, R₉′ is trifluoromethyl.

In some embodiments, the composition is further described as:

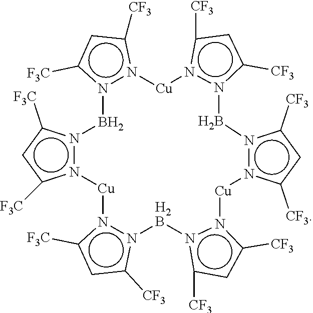

In another aspect, the present disclosure provides a compound of the formula:

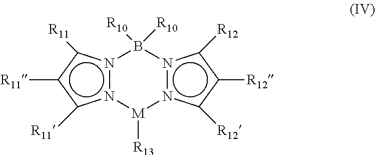

wherein:
R₁₀ and R₁₀′ are each independently selected from hydrogen, alkyl_{(C≤6)}, or substituted alkyl_{(C≤6)};
M is a monovalent metal ion; and
R₁₁, R₁₁′, R₁₂, and R₁₂′ are each independently alkyl_{(C≤12)}, substituted alkyl_{(C≤12)}, aryl_{(C≤12)}, or substituted aryl_{(C≤12)};
R₁₁″ and R₁₂″ is hydrogen, amino, cyano, halo, hydroxy, nitro, alkyl_{(C≤12)}, aryl_{(C≤12)}, acyl_{(C≤12)}, alkoxy_{(C≤12)}, alkylamino_{(C≤12)}, dialkylamino_{(C≤12)}, or a substituted version thereof; and
R₁₃ is ethylene or propylene.

In some embodiments, the compound further defined as:

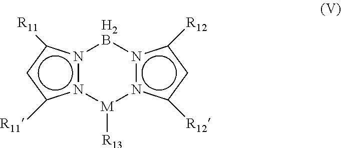

wherein:
M is a monovalent metal ion;
R₁₁, R₁₁′, R₁₂, and R₁₂′ are each independently alkyl_{(C≤12)}, substituted alkyl_{(C≤12)}, aryl_{(C≤12)}, or substituted aryl_{(C≤12)}; and
R₁₃ is ethylene or propylene.

In some embodiments, the compound is further defined as:

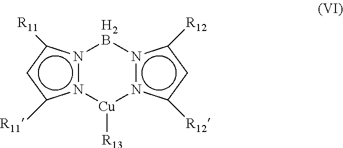

wherein:
R₁₁, R₁₁′, R₁₂, and R₁₂′ are each independently alkyl_{(C≤12)}, substituted alkyl_{(C≤12)}, aryl_{(C≤12)}, or substituted aryl_{(C≤12)}; and
R₁₃ is alkyl_{(C≤12)}, substituted alkyl_{(C≤12)}, alkenyl_{(C≤12)}, or substituted alkenyl_{(C≤12)}.

In some embodiments, R₁₁ is substituted alkyl_{(C≤12)}. In some embodiments, R₁₁ is substituted alkyl_{(C≤6)}. In some embodiments, R₁₁ is trifluoromethyl.

In some embodiments, R₁₁′ is substituted alkyl_{(C≤12)}. In some embodiments, R₁₁′ is substituted alkyl_{(C≤6)}. In some embodiments, R₁₁′ is trifluoromethyl.

In some embodiments, R₁₂ is substituted alkyl_{(C≤12)}. In some embodiments, R₁₂ is substituted alkyl_{(C≤6)}. In some embodiments, R₁₂ is trifluoromethyl.

In some embodiments, R₁₂′ is substituted alkyl_{(C≤12)}. In some embodiments, R₁₂′ is substituted alkyl_{(C≤6)}. In some embodiments, R₁₂′ is trifluoromethyl.

In some embodiments, the compound is further defined as:

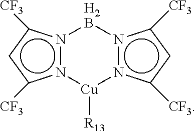

In some embodiments, R₁₃ is ethylene. In some embodiments, R₁₃ is propylene.

In some embodiments, the compound is further defined as an organometallic complex.

In yet another aspect, there is provided a separation device comprising a compound of the present aspects and embodiments thereof.

A further aspect provides a method of separating an alkene_{(C≤12)} or substituted alkene_{(C≤12)} from an alkane_{(C≤12)} or substituted alkane_{(C≤12)} comprising contacting a mixture comprising an alkene_{(C≤12)} or substituted alkene_{(C≤12)} and an alkane_{(C≤12)} or substituted alkane_{(C≤12)} with a compound according to the present aspects and embodiments thereof.

In some embodiments, the mixture is a gas. In some embodiments, the method comprises a step of removing the alkane_{(C≤12)} or substituted alkane_{(C≤12)}. In some embodiments, the alkane_{(C≤12)} or substituted alkane_{(C≤12)} is removed by refluxing the compound in an organic solvent.

In some embodiments, the organic solvent is an alkane_{(C≤8)}. In some embodiments, the organic solvent is hexanes.

In some embodiments, the alkane_{(C≤12)} or substituted alkane_{(C≤12)} is removed under reduced pressure. In some embodiments, the reduced pressure is less than 1 bar.

In some embodiments, the temperature is from about 25° C. to about 75° C. In some embodiments, the temperature is from about 30° C. to about 50° C. In some embodiments, the mixture is contacted with the composition at a temperature from about 0° C. to about 75° C. In some embodiments, the temperature is from about 20°° C. to about 50°° C. In some embodiments, the temperature is from about 20° C. to about 30° C.

In some embodiments, the alkane_{(C≤12)} or substituted alkane_{(C≤12)} is ethane. In some embodiments, the alkene_{(C≤12)} or substituted alkene_{(C≤12)} is ethylene.

In some embodiments, the alkane_{(C≤12)} or substituted alkane_{(C≤12)} is propane. In some embodiments, the alkene_{(C≤12)} or substituted alkene_{(C≤12)} is propylene.

In another aspect, there is provided a method of separating propylene from propane comprising contacting a mixture comprising propylene and propane with a compound of the present aspects and embodiments thereof.

In yet another aspect, there is provided a method of separating ethylene from ethane comprising contacting a mixture comprising ethylene and ethane with a compound of the present aspects and embodiments thereof.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1: Copper(I) ethylene complexes [(CH₃)₂Tp]Cu(C₂H₄) (1) and [(CF₃)₂Tp]Cu(C₂H₄) (2) supported by scorpionates, as well as reversible structural rearrangement of [(CF₃)₂Bp]Cu(C₂H₄) (3) and {[(CF₃)₂Bp]Cu}₃ (4) driven by C₂H₄ removal and sorption.

FIG. 2: Molecular structures of [(CF₃)₂Bp]Cu(C₂H₄) (3) (top) and {[(CF₃)₂Bp]Cu}₃ (4) (bottom). Selected bond distances (Å) and angles)(°) for [(CF₃)₂Bp]Cu(C₂H₄): Cu—N2 1.9873(13), Cu—N4 1.9885(12), Cu—C11 2.0184(16), Cu—C12 2.0182(16), C11-C12 1.360(3), Cu···B 2.9823(19), N2-Cu—N4 93.28(5), C12-Cu—C11 39.38(7); for {[(CF₃)₂Bp]Cu}₃: Cu1-N2 1.8900(17), Cu1-N2ⁱ 1.8900(17), Cu2-N4 1.8955(17), Cu2-N6 1.8942(17); N2-Cu1—N2ⁱ 176.32(10), N6-Cu2-N4 177.46(7), Cu1···B2 5.767(3), Cu1···Cu2 5.8570(6); Cu2···Cu2ⁱ5.8836(6).

FIGS. 3A-3D: (FIG. 3A) Single-component sorption isotherms of ethylene (upper trace), ethane (lower trace) at 298 K for {[(CF₃)₂Bp]Cu}₃; (FIG. 3B) Qualitative comparison of LAST adsorption selectivities of {[(CF₃)₂Bp]Cu}₃ with the benchmark porous material UTSA-280 for an equimolar ethylene/ethane mixture at 298 K; (FIG. 3C) breakthrough curves of {[(CF₃)₂Bp]Cu}₃ for an equimolar binary mixture of C₂H₄/C₂H₆ (50/50 v/v) at 298 K and 100 kPa (1 bar); (FIG. 3D) The recyclability of {[(CF₃)₂Bp]Cu}₃ in multiple breakthrough experiments under the same condition.

FIG. 4: Synthesis of [(CF₃)₂Bp]Cu(C₂H₄) (3).

FIG. 5: Synthesis of {[(CF₃)₂Bp]Cu}₃ (4).

FIG. 6: ¹H NMR Spectrum of [(CF₃)₂Bp]Cu(C₂H₄) (3) in CDCl₃.

FIG. 7: ¹³C NMR Spectrum of [(CF₃)₂Bp]Cu(C₂H₄) (3) in CDCl₃.

FIG. 8: ¹⁹F NMR Spectrum of [(CF₃)₂Bp]Cu(C₂H₄) (3) in CDCl₃.

FIG. 9: Raman Spectrum of [(CF₃)₂Bp]Cu(C₂H₄) (3).

FIG. 10: ¹H NMR Spectrum of {[(CF₃)₂Bp]Cu}₃ (4) in CDCl₃.

FIG. 11: ¹H NMR Spectrum of {[(CF₃)₂Bp]Cu}₃ (4) in CD₂Cl₂.

FIG. 12: ¹H NMR Spectrum of {[(CF₃)₂Bp]Cu}₃ (4) in (CD₃)₂SO.

FIG. 13: ¹³C NMR Spectrum of {[(CF₃)₂Bp]Cu}₃ (4) in (CD₃)₂SO (DMSO was used just for ¹³C and ¹⁹F NMR due to poor solubility in CDCl₃ and CD₂Cl₂).

FIG. 14: ¹⁹F NMR Spectrum of {[(CF₃)₂Bp]Cu}₃ (4) in (CD₃)₂SO (DMSO was used just for ¹³C and ¹⁹F NMR due to poor solubility in CDCl₃ and CD₂Cl₂).

FIG. 15: Comparison of Raman Spectra of {[(CF₃)₂Bp]Cu}₃ (4) and [(CF₃)₂ Bp]Cu(C₂H₄) (3).

FIG. 16: X-ray crystal structure of [(CF₃)₂Bp]Cu(C₂H₄) (3). Two views are given.

FIG. 17: X-ray crystal structure of {[(CF₃)₂Bp]Cu}₃ (4) (top). Two views of 18-membered, Cu₃N₁₂B₃ core (bottom).

FIG. 18: Crystal packing diagram of [(CF₃)₂Bp]Cu(C₂H₄) (3). There are no empty spaces (voids), based on the calculations using CCDC Mercury 2020.3.0 using a probe radius of 1.2 Å and approximate grid spacing of 0.7 Å.

FIG. 19: Crystal packing diagram of {[(CF₃)₂Bp]Cu}₃ (4) showing empty spaces (voids), which was calculated using CCDC Mercury 2020.3.0 using a probe radius of 1.2 Å and approximate grid spacing of 0.7 Å, came to 699 Å³ (13.1% unit cell volume). Calculation of voids using 1.65 Å probe radius (considering smallest dimension of ethylene based on estimated size of ethylene 3.28×4.18×4.84 Å³) gave a void volume of 621 Å³ (11.7% unit cell volume). As noted in the experimental section, these voids are occupied by disordered hexane in the crystal, which could not be modeled and was treated and removed with Olex2 solvent mask command.

FIGS. 20A-20B: Langmuir-Freundlich fitting of the C₂H₄ (FIG. 20A) and C₂H₆ (FIG. 20B) sorption data at 298 K for {[(CF₃)₂Bp]Cu}₃ (4).

FIG. 21: Schematic illustration of the apparatus for the breakthrough experiments.

FIG. 22: The calculation for captured amount of C₂H₄ during the breakthrough process in {[(CF₃)₂Bp]Cu}₃ (4). During the duration before the breakthrough point (0-t₁), the captured C₂H₄ is 0.7 mmol, corresponding to 0.60 mmol/g. Considering the continuous C₂H₄ adsorption during the mass transfer zone (t₁-t₂), the integration of the grey area above the entire breakthrough curve gave the maximum loading of the sample to be 0.83 mmol, corresponding to 0.72 mmol/g.

FIG. 23: Multiple cycles of breakthrough curves for equimolar binary mixture of C₂H₄/C₂H₆ at 298 K and 1 bar. The breakthrough experiments were carried out in a packed column at a flow rate of 2.0 mL min⁻¹. Points are experimental data, and lines are drawn to guide the eye.

FIG. 24: Top view of the powder diffraction patterns as [(CF₃)₂Bp]Cu(C₂H₄) converts to the ethylene free sorbent under slow heating and a He flow. The bottom section of the figure shows the powder diffraction pattern of pure [(CF₃)₂Bp]Cu(C₂H₄). The top section of the figure shows the powder diffraction pattern for the ethylene free copper complex.

FIG. 25: Top view of the powder diffraction patterns for the ethylene loading experiment of the in-situ generated sorbent. This shows the rapid conversion to the [(CF₃)₂Bp]Cu(C₂H₄) complex as soon as ethylene was introduced.

FIG. 26: Schematic depicting method for production of >99.5% pure ethylene.

FIG. 27: Synthesis of [(CF₃)₂Bp]Cu(C₃H₆).

FIG. 28: Synthesis of {[(CF₃)₂Bp]Cu}₃ by removing propylene from [(CF₃)₂Bp]Cu(C₃H₆).

FIG. 29: Breakthrough curves of {[(CF₃)₂Bp]Cu}₃ for an equimolar binary mixture of C₃H₆/C₃H₈ (50/50 v/v) at 298 K and 100 kPa (1 bar); and the recyclability of {[(CF₃)₂Bp]Cu}₃ in multiple breakthrough experiments under the same conditions.

FIG. 30: The calculation for captured amount of C₃H₆ during the breakthrough process in {[(CF₃)₂Bp]Cu}₃ (4). During the duration before the breakthrough point (0-t₁), the captured C₃H₆ was 0.51 mmol, corresponding to 0.44 mmol/g. Considering the continuous C₃H₆ adsorption during the mass transfer zone (t₁-t₂₎, the integration of the grey area above the entire breakthrough curve gave the maximum loading of the sample to be 0.53 mmol, corresponding to 0.46 mmol/g.

FIGS. 31A-31B:Single-component sorption isotherms of C₃H₆ uptake at 298K and 100 kPa (1 bar) for {[(CF₃)₂Bp]Cu}₃ (4). The sample was reactivated under 40° C. for another 12 h before the C₃H₆ measurement. It was found that the maximum adsorption amount of C₃H₆ at 298 K and 100 kPa (1 bar) is 36.5 cm³/g (FIGS. 31A), which is slightly smaller than that of C₂H₄ uptake (39.4 cm³/g) under the same conditions. Interestingly, after reactivated under RT. for 6 h, the adsorption of the sample at 298K and 100 kPa (1 bar) toward C₃H₆ recovered (FIG. 31B).

FIG. 32: Single-component sorption isotherms of C₃H₆ uptake at 273K and 100 kPa (1 bar) for {[(CF₃)₂Bp]Cu}₃ (4). The sample uptakes less amount (30.2 cm³/g) of C₃H₆ at 273 K and 100 kPa (1 bar).

FIGS. 33: Single-component sorption isotherms of C₃H₈ uptake at 298K and 100 kPa (1 bar) for {[(CF₃)₂Bp]Cu}₃ (4). The sample has almost no adsorption (0.31 cm³/g) toward C₃H₈ at 298K and 100 kPa (1 bar).

FIGS. 34A-34B: Langmuir-Freundlich fitting of the C₃H₆ (FIG. 34A) and C₃H₈ (FIG. 34B) sorption data at 298 K for {[(CF₃)₂Bp]Cu}₃ (4).

FIG. 35: Selectivity of {[(CF₃)₂Bp]Cu}₃ (4) for C₃H₆/C₃H₈ (50:50) at 298 K predicted by IAST.

### DESCRIPTION OF ILLUSTRATIVE EMBODIMENTS

Purification of C₂H₄ from a C₂H₄/C₂H₆ mixture is a difficult separation process, which is achieved mainly through energy-intensive, cryogenic distillation in industry. Sustainable, non-distillation methods are desired as alternatives. Provided herein fluorinated bis(pyrazolyl)borate ligand supported copper(I) complex {[(CF₃)₂Bp]Cu}₃ has features desirable in an olefin-paraffin separation material. It binds ethylene exclusively over ethane generating [(CF₃)₂Bp]Cu(C₂H₄). This molecular sorbent exhibits extremely high and record IAST C₂H₄/C₂H₆ gas separation selectivity, affording high purity (>99.5%) ethylene that can be readily desorbed from separation columns. In-situ PXRD provides a “live” picture of the reversible conversion between [(CF₃)₂Bp]Cu(C₂H₄) and the ethylene free sorbent in the solid- state driven by the presence or removal of C₂H₄. Crystal structures of trinuclear {[(CF₃)₂Bp]Cu}₃ and mononuclear [(CF₃)₂Bp]Cu(C₂H₄) are also provided herein.

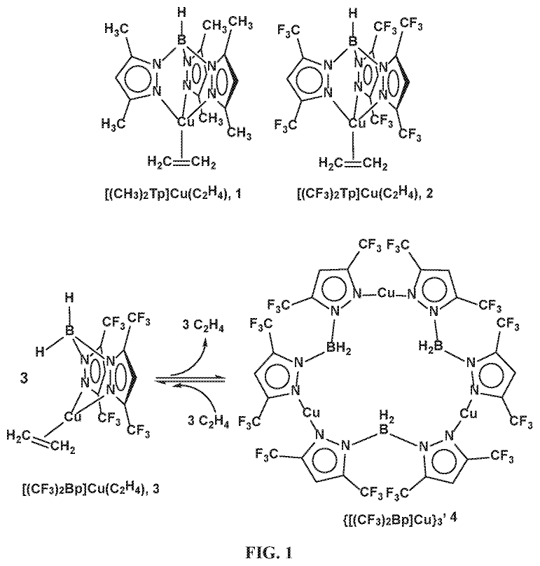

In certain aspects, the present disclosure provides a molecular sorbent based on copper and a popular ligand class that provides high-purity ethylene from ethylene-ethane mixtures and high-purity propylene from propylene-propane mixtures. Although [(CF₃)₂Tp]Cu(C₂H₄) (2) is too inert for this purpose, the inventors discovered that the fluorinated bis(pyrazolyl)borate [(CF₃)₂Bp]Cu(C₂H₄) (3) (based on a ligand that is missing a pyrazolyl arm from 2, FIG. 1) has can be used for effective ethylene-ethane separation as well as propylene- propane separation. Thus, further provided herein are metal organic complexes or organometallic complexes [(CF₃)₂Bp]Cu(C₂H₄) and [(CF₃)₂Bp]Cu(C₃H₆) and methods of use thereof.

### I. Definitions

A “molecular sorbent” is a molecular species used to recover liquids or gases through the mechanism of absorption, or adsorption, or both.

A “metal organic complex” or “organometallic complex” is used herein to refer to a compound with least one metal-to-carbon bond in which the carbon is part of an organic group.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Additionally, it is contemplated that one or more of the metal atoms may be replaced by another isotope of that metal. In some embodiments, the calcium atoms can be ⁴⁰Ca, ⁴²Ca, ⁴³Ca, ⁴⁴Ca, ⁴⁶Ca, or ⁴⁸Ca. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Any undefined valency on a carbon atom of a structure shown in this application implicitly represents a hydrogen atom bonded to the atom.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

The term “saturated” when referring to an atom means that the atom is connected to other atoms only by means of single bonds.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means 'N₃; in a monovalent context “phosphate” means —OP(O)(OH)₂ or a deprotonated form thereof, in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “- - - -” represents an optional bond, which if present is either single or double. The symbol “ [symbol] ” represents a single bond or a double bond. Thus, the formula

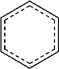

covers, for example,

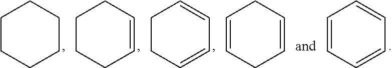

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

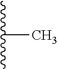

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

The bond orders described above are not limiting when one of the atoms connected by the bond is a metal atom (M). In such cases, it is understood that the actual bonding may comprise significant multiple bonding and/or ionic character. Therefore, unless indicated otherwise, the formulas M—C, M═C, M - - - - C, and M [symbol] C, each refers to a bond of any and type and order between a metal atom and a carbon atom.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8})”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C≤12)} group; however, it is not an example of a dialkylamino_{(C6)} group. Likewise, phenylethyl is an example of an aralkyl_{(C≤8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto- enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon- carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

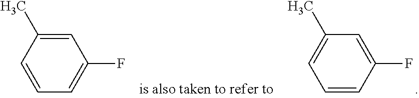

Aromatic compounds may also be depicted using a circle to represent the delocalized nature of the electrons in the fully conjugated cyclic π system, two non-limiting examples of which are shown below:

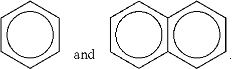

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

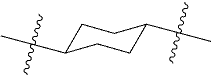 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH—CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon- carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

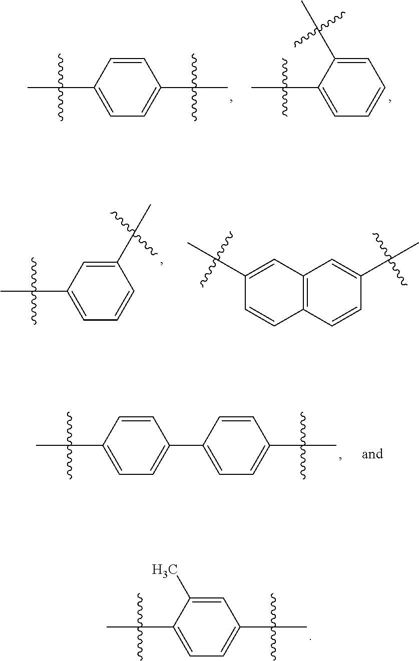

An “arene” refers to the class of compounds having the formula H—R, wherein R is aryl as that term is defined above. Benzene and toluene are non-limiting examples of arenes.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃); (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: CH₂OH, —CH₂Cl, —CF₃, —CH₂CN, —CH₂C(O)OH, —CH₂C(O)OCH₃—CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups. “Metal-organic frameworks” (MOFs) are framework materials, typically three-dimensional, self-assembled by the coordination of metal ions with organic linkers exhibiting porosity, typically established by gas adsorption. The MOFs discussed and disclosed herein are at times simply identified by their repeat unit as defined below without brackets or the subscript n. A mixed-metal-organic frameworks (M′MOF) is a subset of MOFs having two of more types of metal ions.

The term “unit cell” is basic and least volume consuming repeating structure of a solid. The unit cell is described by its angles between the edges (α, β, γ) and the length of these edges (a, b, c). As a result, the unit cell is the simplest way to describe a single crystal X-ray diffraction pattern.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined, it simply designates repetition of the formula within the brackets as well as the polymeric and/or framework nature of the material. The concept of a repeat unit applies equally to where the connectivity between the repeat units extends into three dimensions, such as in metal organic frameworks, cross-linked polymers, thermosetting polymers, etc. Note that for MOFs the repeat unit may also be shown without the subscript n.

“Pores” or “micropores” in the context of metal-organic frameworks are defined as open space within the MOFs; pores become available, when the MOF is activated for the storage of gas molecules. Activation can be achieved by heating, e.g., to remove solvent molecules.

“Multimodal size distribution” is defined as pore size distribution in three dimensions.

“Multidentate organic linker” is defined as ligand having several binding sites for the coordination to one or more metal ions.

The above definitions supersede any conflicting definition in any of the reference that is incorporated herein by reference. The fact that certain terms are defined, however, should not be considered as indicative that any term that is undefined is indefinite. Rather, all terms used are believed to describe the invention in terms such that one of ordinary skill can appreciate the scope and practice the present invention.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Synthesis and Characterization of Molecular Sorbent

It was discovered that the fluorinated bis(pyrazolyl)borate [(CF₃)₂Bp]Cu(C₂H₄) (3) (based on a ligand that is missing a pyrazolyl arm from 2, FIG. 1) has exceptional features for effective ethylene-ethane and propylene-propane separation.

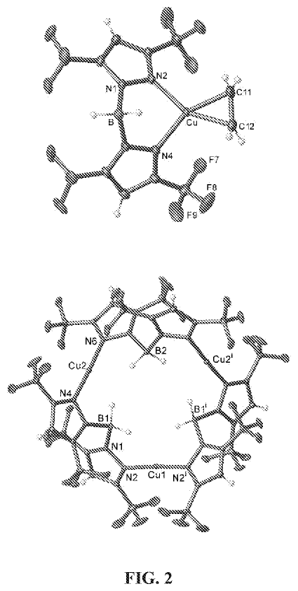

Treatment of [(CF₃)₂Bp]Cu(NCMe) (Dias et al., 2005) with ethylene in dichloromethane at room temperature led to the displacement of acetonitrile ligand from copper and the formation of copper(I) ethylene complex [(CF₃)₂Bp]Cu(C₂H₄) (3) in 92% isolated yield. It is a colorless, thermally stable solid that can be handled in air for hours without decomposition. The ethylene protons of 3 in the ¹H NMR spectrum appear at δ 4.69 ppm. This points to an intermediate level of olefinic proton shielding relative to those of 1 (δ 4.41 ppm) and 2 (δ 4.96 ppm). The presence of additional ethylene leads to separate broad signals of free and coordinated ethylene indicating associative olefin exchange. In contrast, coordinatively saturated 2 does not exchange with free ethylene at room temperature. The ¹³C{¹H} NMR spectrum of [(CF₃)₂Bp]Cu(C₂H₄) (3) in CDCl₃ shows a peak at δ 86.6 ppm corresponding to the carbons of the copper bound ethylene moiety, which is an upfield shift (of 36.5 ppm) compared to the corresponding resonance of the free C₂H₄, δ 123.1 ppm. The υ̅(C═C) of solid 3 was observed at 1539 cm⁻¹ in the Raman spectrum. For comparison, free ethylene exhibits the C═C stretch coupled with the CH₂ scissoring vibrations at 1623 cm⁻¹ (Maslowsky, 2019). Molecular structure of 3 was unambiguously established by single-crystal X-ray diffraction (FIG. 2). It is a three-coordinate copper complex with an η²-bound C₂H₄ moiety. The bis(pyrazolyl)borate ligand adopts the familiar boat conformation.

More importantly, it is possible to remove the coordinated C₂H₄ from solid 3 using reduced pressure with mild heat (˜40° C.) or by refluxing a solution of 3 in hexanes. The resulting ethylene-free product {[(CF₃)₂Bp]Cu}₃ (4) converts readily back to 3 upon treatment with C₂H₄ (1 atm) in solution (FIG. 1). The compound {[(CF₃)₂Bp]Cu}₃ (4) was crystallized from hexane and the structure was established by X-ray crystallography. It is a quite interesting, trinuclear species with a metallacrown (18-MC-3) structure (Mezei et al., 2007), featuring bridging bis(pyrazolyl)borates. Any such structures involving bis(pyrazolyl)borate ligands are not known. Compound {[(CF₃)₂Bp]Cu}₃ (4) sits on a two-fold rotation axis. Two- coordinate copper sites adopt a linear geometry.

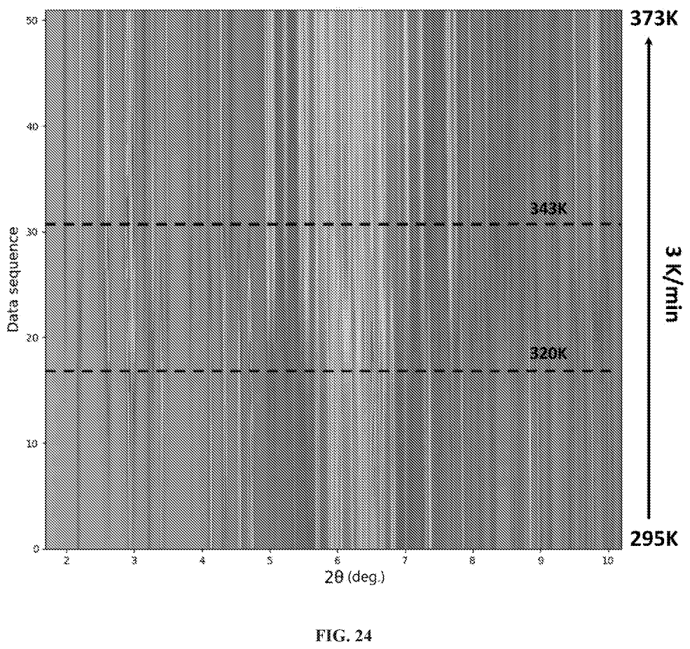

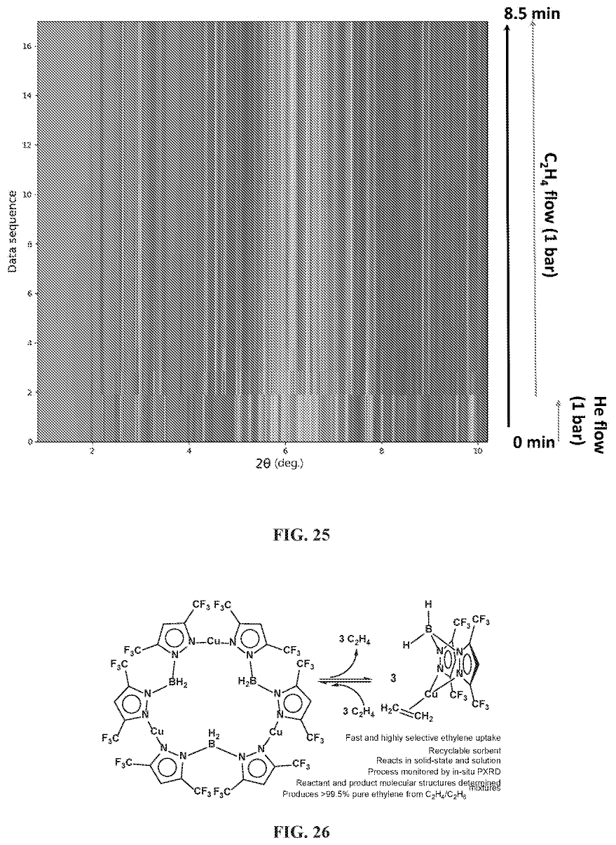

Remarkably, the ethylene-driven transformation of copper(I) sorbent to mononuclear [(CF₃)₂Bp]Cu(C₂H₄) also occurs in dense crystalline materials in the solid-state. To confirm this, in-situ PXRD measurements were performed at 17-BM beamline at the Advanced Photon Source, Argonne National Laboratory. It is possible to remove ethylene from [(CF₃)₂Bp]Cu(C₂H₄) in a flow of helium under gentle heating, and generate the ethylene free copper sorbent (FIG. 24). Then, under ethylene flow at 100 kPa (1 bar) and at 295 K, there is almost instant and complete conversion of this ethylene free sorbent into [(CF₃)₂Bp]Cu(C₂H₄) (FIG. 25). This ethylene-induced conversion (FIG. 1), and its reversible nature in the solid-state was observed by in-situ PXRD.

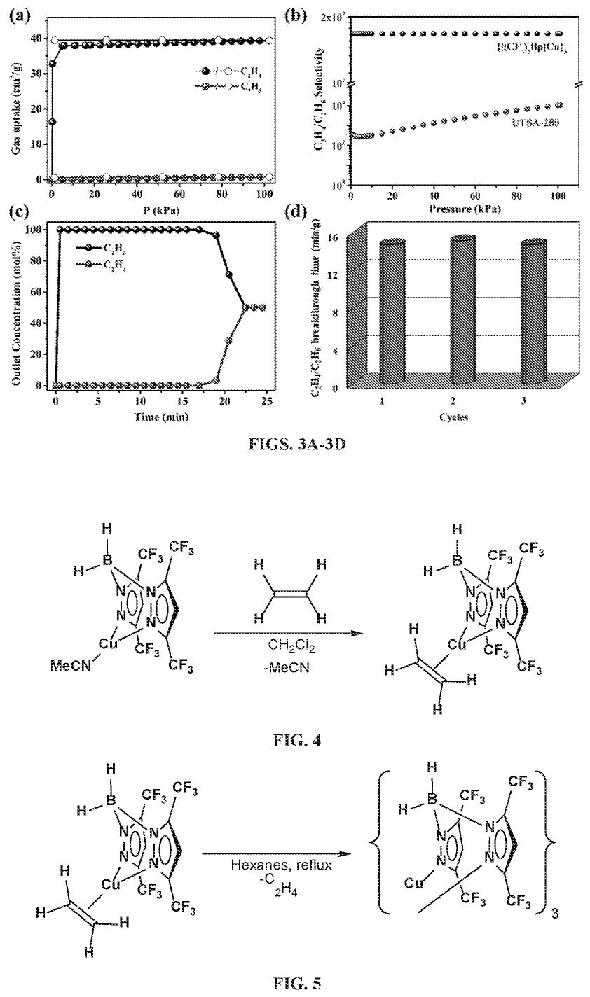

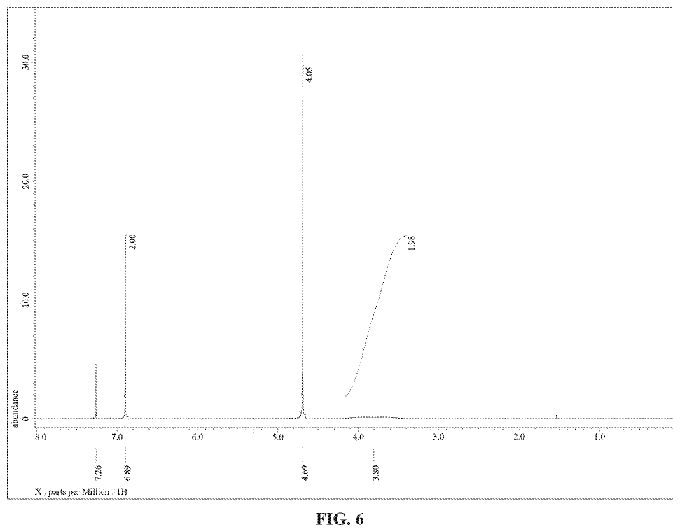

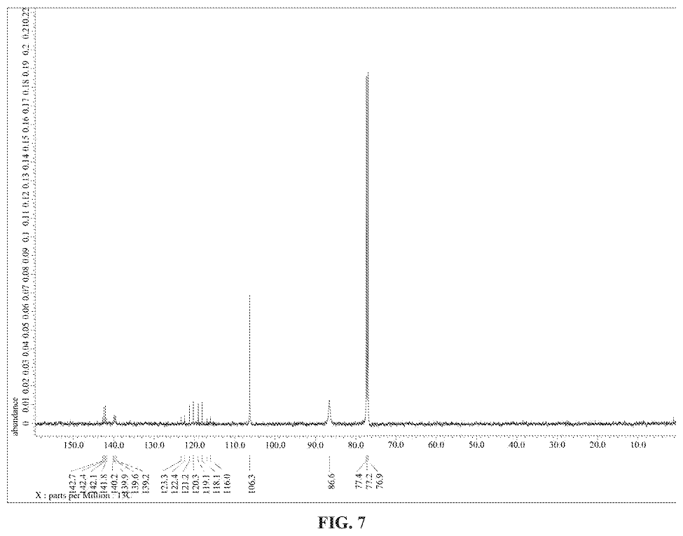

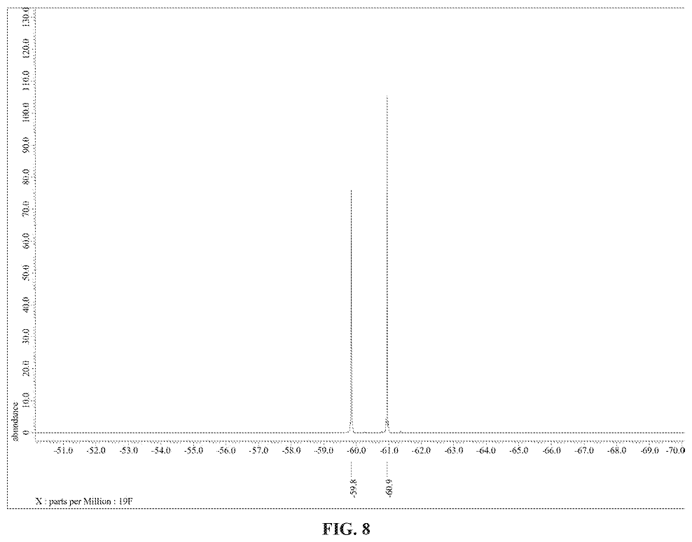

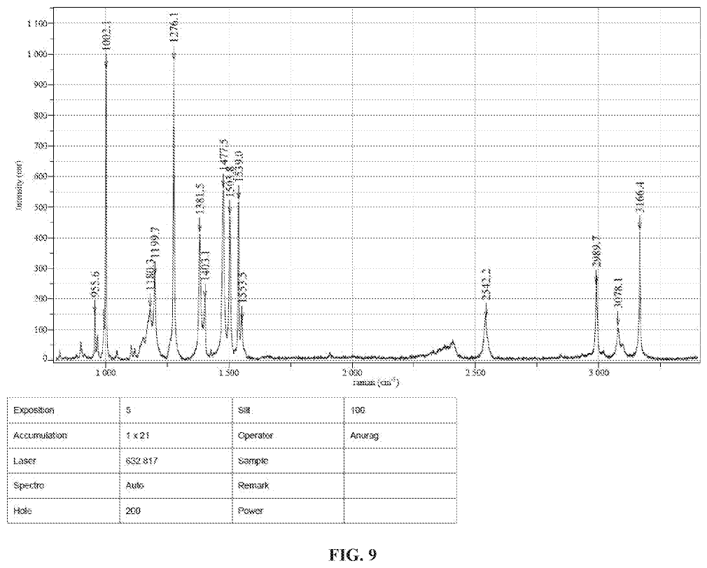

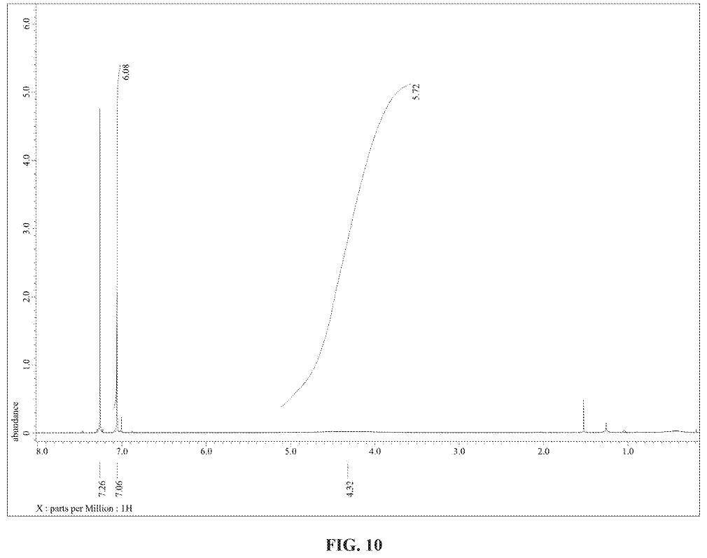

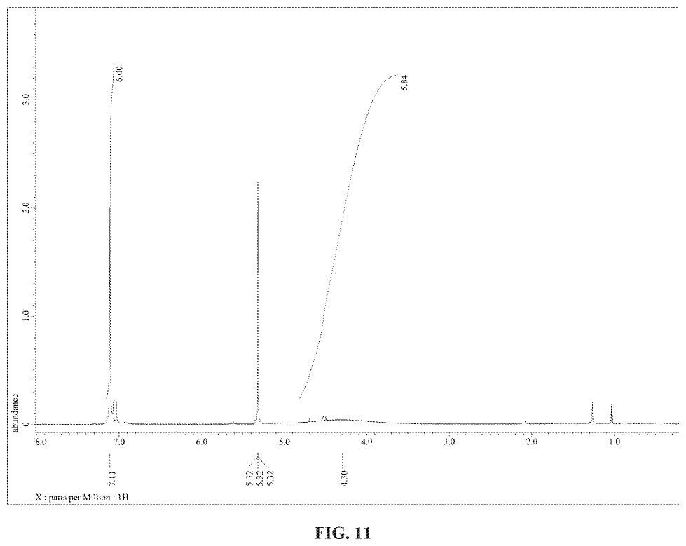

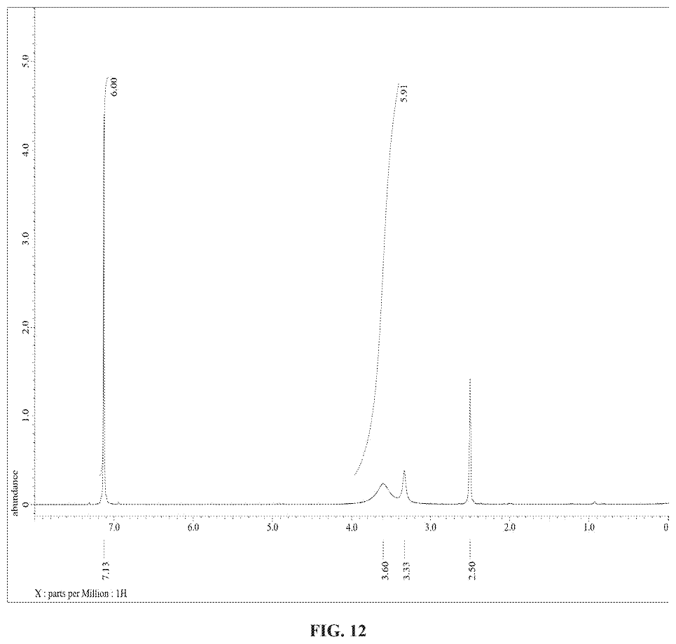

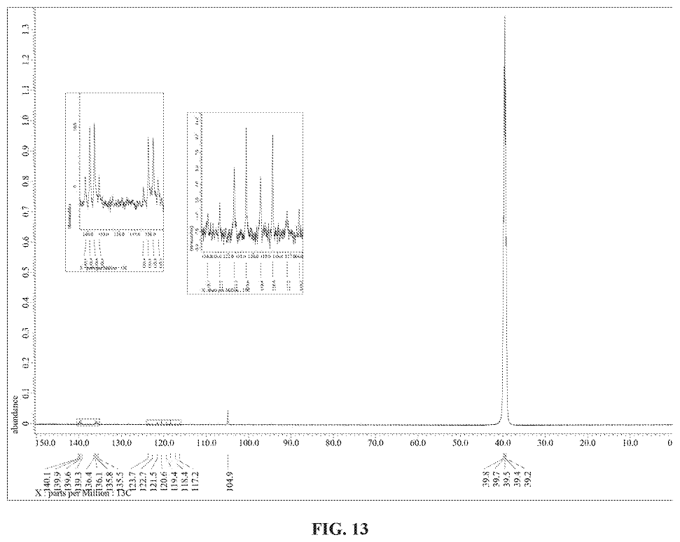

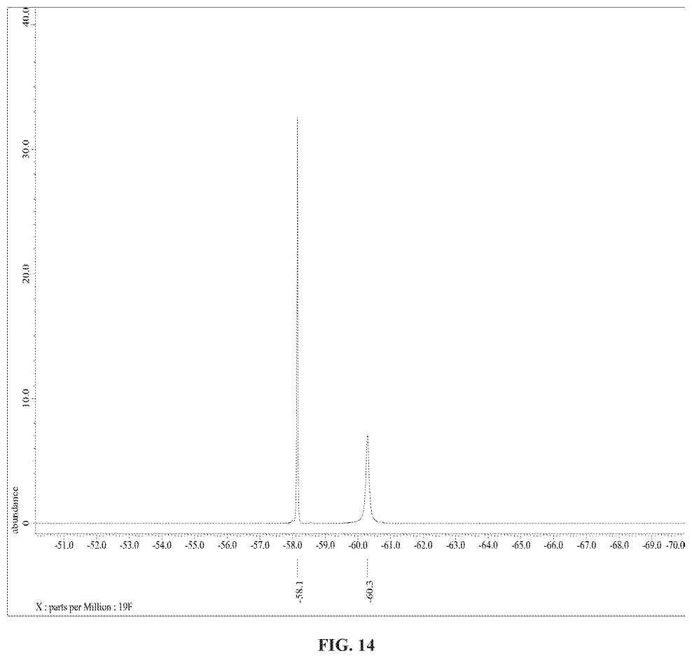

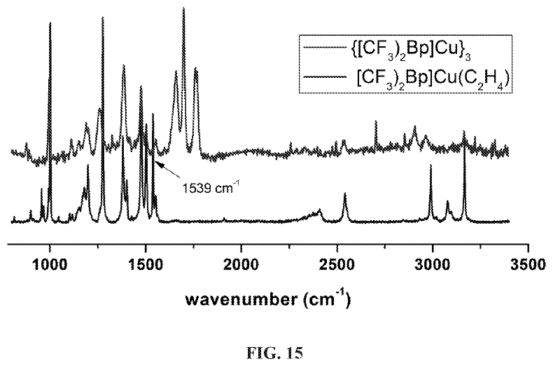

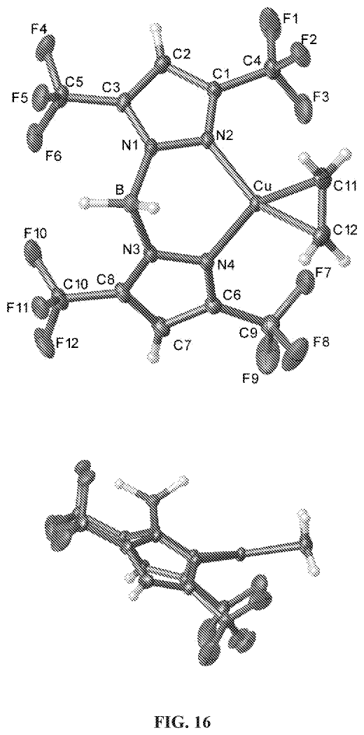

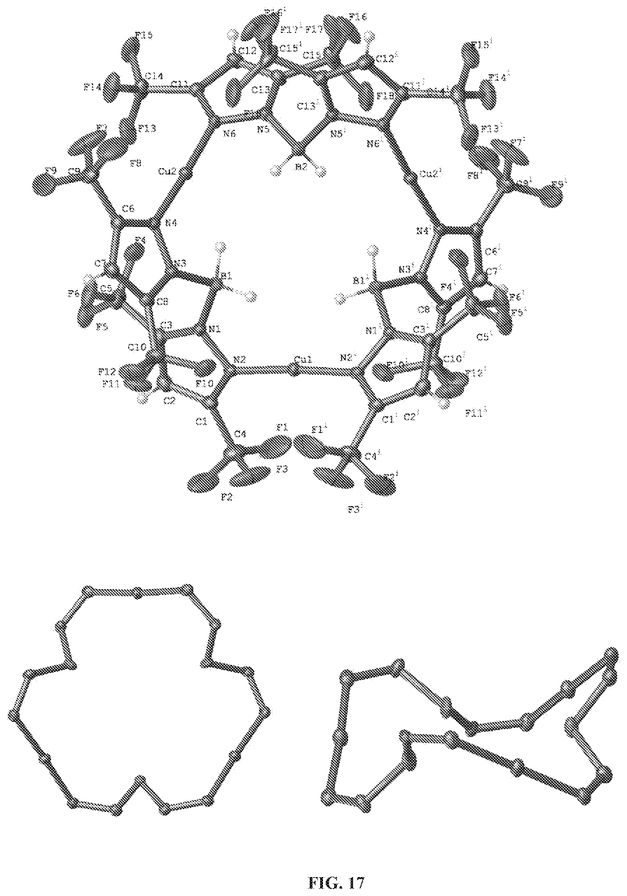

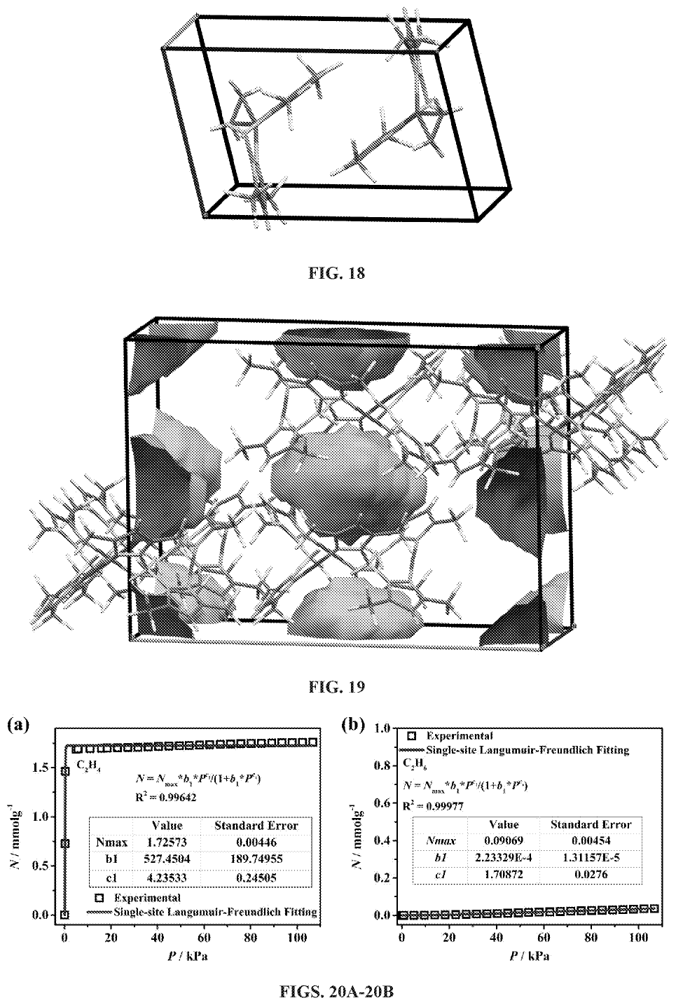

To evaluate the adsorptive separation of C₂H₄ and C₂H₆ on {[(CF₃)₂Bp]Cu}₃, single-component adsorption isotherms of the two gases were collected at 298 K. It was shown that the adsorption isotherm for C₂H₄ on {[(CF₃)₂Bp]Cu}; exhibited a steep slope at relatively low pressure, indicating a strong affinity of {[(CF₃)₂Bp]Cu}₃ towards C₂H₄ molecules (FIG. 3A). At 298 K and 100 kPa (1 bar), the C₂H₄ uptake by {[(CF₃)₂Bp]Cu}₃ reaches 39.4 cm³ g⁻¹ (1.8 mmol g⁻¹); in contrast, {[(CF₃)₂Bp]Cu}₃ adsorbs a negligible amount of C₂H₆ (0.8 cm³ g⁻¹) under the same conditions. From the measured isotherms, a record high C₂H₄/C₂H₆ ideal absorbed solution theory (IAST) selectivity at 298 K and 100 kPa, exceeding 1.7×10⁷, can be estimated. The Cu(I) in {[(CF₃)₂Bp]Cu}₃ exclusively binds ethylene over ethane; while those reported porous materials simultaneously adsorb both ethylene and ethane through the pores that have a certain degree of sieving effects, thus the IAST C₂H₄/C₂H₆ separation selectivity is significantly higher than those reported (Lin et al., 2018), FeMOF-74 (13.6) (Geier et al., 2013), NOTT-300 (48.7) (Yang et al., 2015), PAF-1-SO₃Ag (27) (Li et al., 2014), CuI@UiO-66—(COOH)₂ (80.8) (Zhang et al., 2020), Co-gallate (52) (Bao et al., 2018), HOF-4a (14) (Li et al., 2014). A comparison with the best reported porous material UTSA-280 (>10000) (Lin et al., 2018) is shown in FIG. 3B.

In a typical ethylene production process based on the cracking of heavier hydrocarbon fractions followed by dehydrogenation reactions, resulting in conversion yield of only around 50-60% (Faiz et al., 2012). Thus, the separation of C₂H₄ from the C₂H₄/C₂H₆ mixture is needed before further utilization. To evaluate the performance of {[(CF₃)₂Bp]Cu}₃ in an adsorptive dynamic separation process, breakthrough experiments were performed, in which an equimolar C₂H₄/C₂H₆ mixture flowed over a packed column of the activated {[(CF₃)₂Bp]Cu}₃ solid with a rate of 2 mL min⁻¹ at 298 K (FIG. 3C, 21). As expected, a clean separation of the C₂H₄/C₂H₆ mixture was achieved: C₂H₆ was first to elute through the bed, and the outlet gas quickly reached pure grade with no detectable C₂H₄, which results in a high concentration of C₂H₄ feed that was >99.5% pure (FIG. 3C). The solid adsorbent retained C₂H₄ for an adequate time before its breakthrough. Therefore, C₂H₆ can be removed from C₂H₄ with no loss of valuable C₂H₄, which is in line with the sorption experiments. The amount of C₂H₄ enriched from the equimolar C₂H₄/C₂H₆ mixture is 0.72 mol kg⁻¹ (FIG. 22). Moreover, the regeneration of {[(CF₃)₂Bp]Cu}₃ in a vacuum oven at 313 K revealed that the adsorbed gas could be completely recovered within 24 h (FIG. 3D, 23).

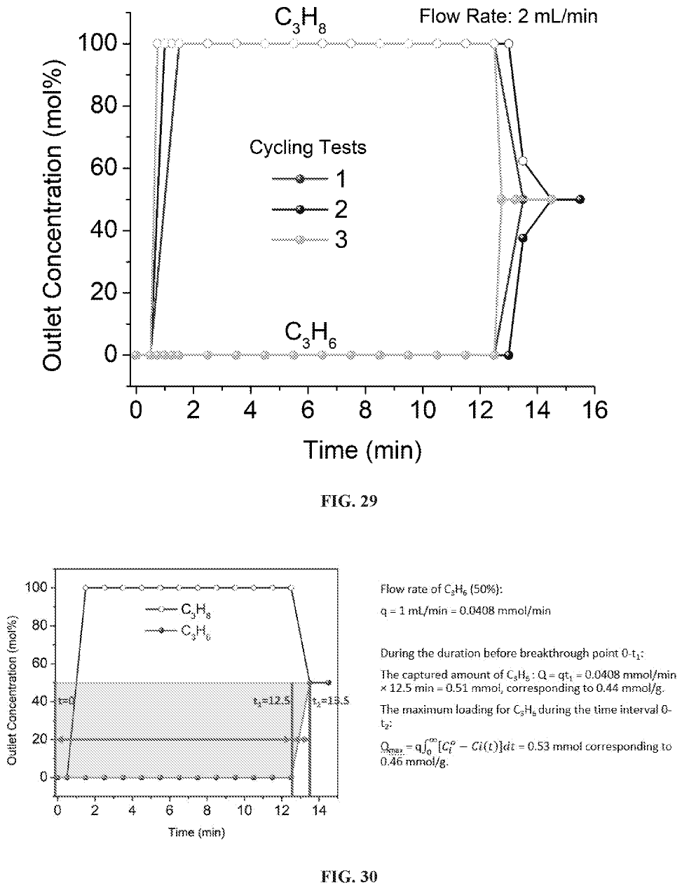

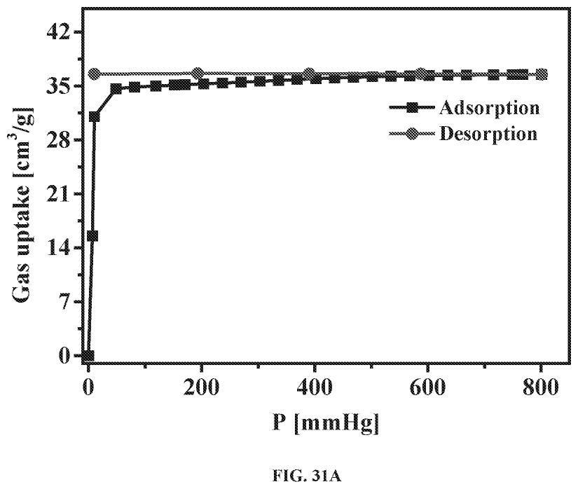

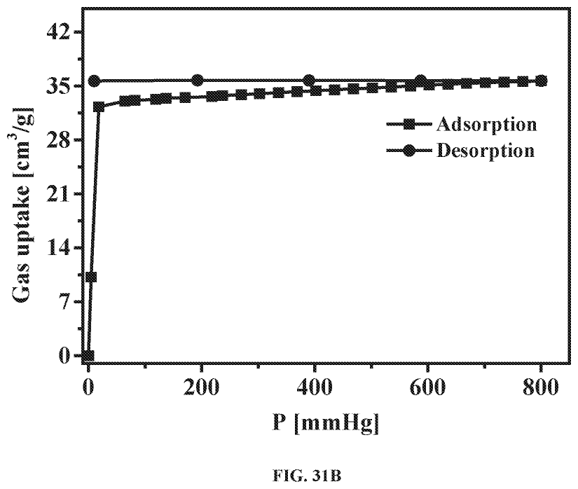

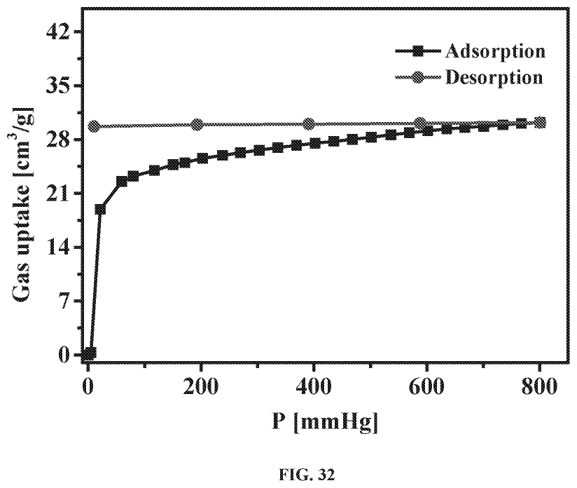

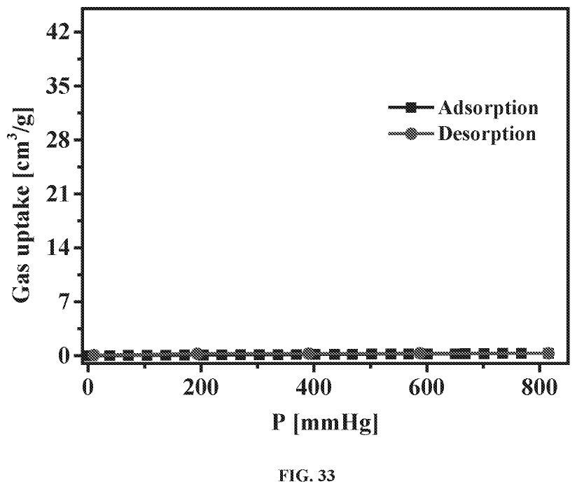

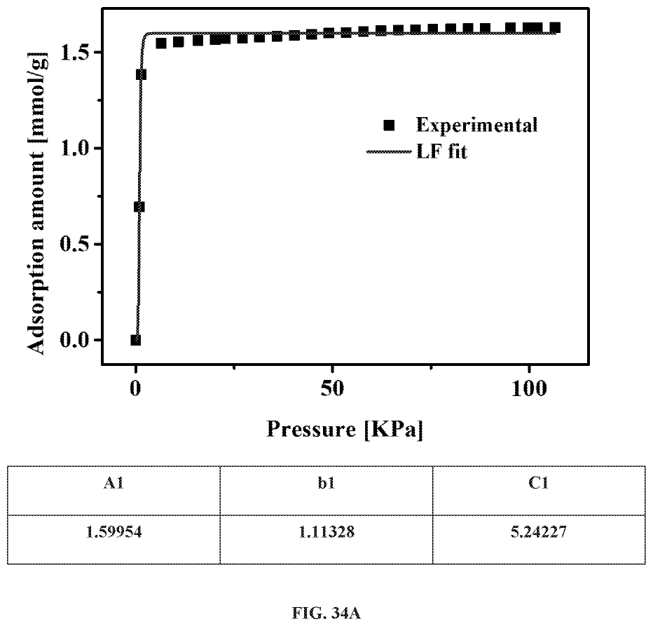

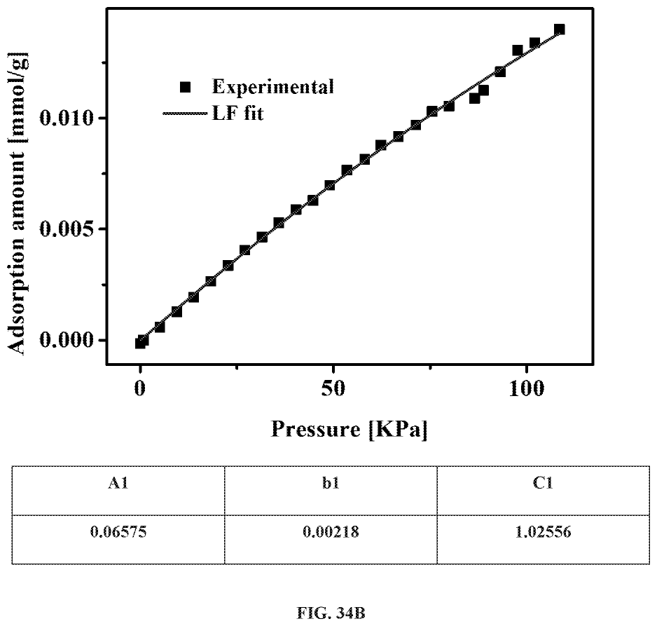

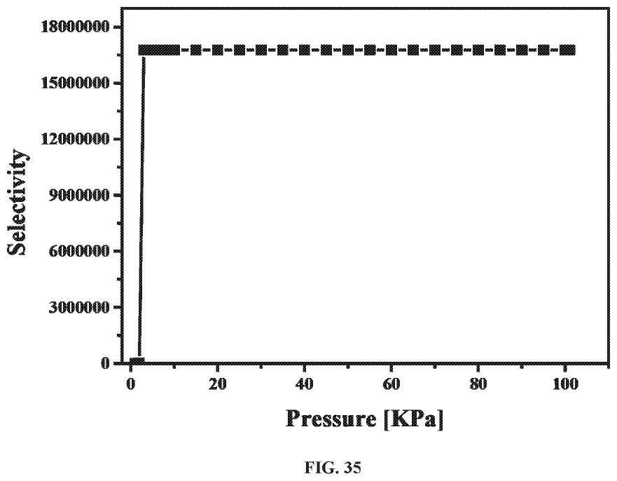

Further, C₃H₆ capture capability was assessed by breakthrough experiments with a C₃H₆/C₃H₈ (50:50, V:V) gas mixture through a packed column with the activated sample under ambient conditions with a flow rate of 2.0 mL/min (FIG. 29-30). From FIG. 29-30, it is revealed that neat separation of C₃H₆ from C₃H₈ by the activated sample could be successfully achieved. From the column outlet, C₃Hs eluted at the very beginning of the process, indicating complete no adsorption of C₃H₈ in the column whereas C₃H₆ underwent a long retention time of ˜12.5 min. On the basis of the breakthrough result, the dynamic C₃H₆ capture capacity of the activated sample for C₃H₆/C₃H₈ (50:50, V:V) mixtures was 0.46 mol/kg. The C₃H₆ capture capacities and separation performances demonstrated by cycling experiments remain steady after several cycles. In the interval of each cycle, the adsorbent was regenerated by a helium flow (40 mL/min) for 2 hours to guarantee a complete removal of the adsorbed gases. During the duration before the breakthrough point (0-t₁), the captured C₃H₆ is 0.51 mmol, corresponding to 0.44 mmol/g. Considering the continuous C₃H₆ adsorption during the mass transfer zone (t₁-t₂), the integration of the grey area above the entire breakthrough curve gave the maximum loading of the sample to be 0.53 mmol, corresponding to 0.46 mmol/g (FIG. 30).

In summary, an effective, molecular sorbent {[(CF₃)₂Bp]Cu}₃ has been uncovered for C₂H₄/C₂H₆ and C₃H₆/C₃H₈ separation. The ethylene uptake was investigated by {[(CF₃)₂Bp]Cu}₃ and removal from [(CF₃)₂Bp]Cu(C₂H₄) in solution, as well as in the solid-state. Similarly, the propylene uptake was investigated by {[(CF₃)₂Bp]Cu}₃ and removal from [(CF₃)₂Bp]Cu(C₃H₆) in solution. Complete characterization of these interesting molecular species including their crystal structures has been achieved. Furthermore, in-situ PXRD provides a complete “live” picture of the solid-state process that also retains the precursor and product crystallinity and undergoes fast and complete and reversible conversion between the copper based sorbent and [(CF₃)₂Bp]Cu(C₂H₄) driven by the presence or removal of C₂H₄. The accessible Cu(I) sites within this molecular compound have enabled it to exclusively bind ethylene over ethane, so this molecular sorbent exhibits extremely high and record IAST C₂H₄/C₂H₆ gas separation selectivity. The superior ethylene separation and purification performance of this molecular sorbent have been further confirmed by the breakthrough experiments in which ethylene of a purity level of over 99.5% can be readily desorbed from the separation columns. Similarly, it was shown that neat separation of C₃H₆ from C₃H₈ by the activated sample could be successfully achieved.

### Example 2: Synthesis and Characterization of Metal Complexes

[(CF₃)₂Bp]Cu(C₂H₄) (3): (FIG. 4) [(CF₃)₂Bp]Cu(NCMe)(1) (0.15 g, 0.29 mmol) was dissolved in 5 mL dichloromethane and stirred for ˜3-5 min while bubbling ethylene. The reaction mixture was concentrated with continuous flow of ethylene and kept at −20 C. to obtain X-ray quality colorless crystals of [(CF₃)₂Bp]Cu(C₂H₄). Yield: 92%. M.P.: 99-105° C. (decomposition). Anal. Calc. C₁₂H₈BCuF₁₂N₄: C, 28.23; H, 1.58%; N, 10.97%. Found: C, 27.97%; H, 1.78%; N, 11.28%. ¹H NMR (CDCl₃): δ (ppm) 6.89 (s, 2H, PzH), 4.69 (s, 4H, C₂H₄), 3.80 (br, 2H, BH₂). ¹⁹F NMR (CDCl₃): δ (ppm) −59.8 (s), −60.9 (s). ¹³C{¹H} NMR (CDCl₃): δ (ppm) 142.2 (q, 2J_{C-F}=39.6 Hz, C-3/C-5), 139.7 (q, ²J_{C-F}=42.0 Hz, C-3/C-5), 120.1 (q, ¹J_{C-F}=268.7 Hz, CF₃), 119.2 (q, ¹J_{C-F}32269.9 Hz, CF₃), 106.3 (C-4), 86.6 (br, C═C). IR (cm⁻¹): 2540, 2419, 1552, 1501, 1426, 1399, 1254, 1174, 1139, 1115, 1099, 1043, 1018, 1003, 960, 954, 897, 828. Raman (cm⁻¹), selected peaks: 1539 (C═C).

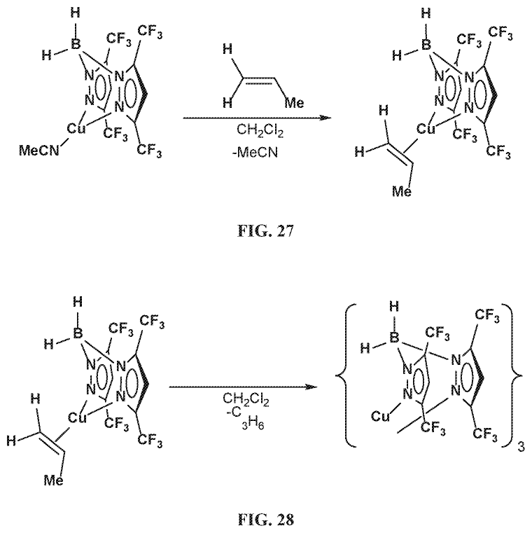

{[(CF₃)₂Bp]Cu}₃ (4): (FIG. 5) [(CF₃)₂Bp]Cu(C₂H₄)(3) (0.15 g, 0.29 mmol) was dissolved in 5 mL hexanes and the solvent was distilled off using a short path distillation apparatus by heating in an oil bath. The product was dissolved in minimum amount of hexanes and kept at −20° C. refrigerator to obtain x-ray quality colorless crystals of {[(CF₃)₂Bp]Cu}₃. Yield: 87%. M.P .: 126-129°° C. (decomposition). Anal. Calc. C₃₀H₁₂B3Cu3F36N12: C, 24.89%; H, 0.84%; N, 11.61%. Found: C, 24.64%; H, 1.24%; N, 11.31%. ¹H NMR ((CD₃)₂SO): δ(ppm) 7.13 (s, 6H, PzH), 3.60 (br, 6H, BH₂). ¹H NMR (CDCl₃): δ (ppm) 7.06 (s, 6H, PzH), 4.32 (br, 6H, BH₂). ¹H NMR ((CD₂Cl₂): δ (ppm) 7.11 (s, 6H, PzH), 4.30 (br, 6H, BH₂). ¹⁹F NMR ((CD₃)₂SO): δ(ppm) −58.1 (s), −60.3 (s). ¹³C{¹H} NMR ((CD₃)₂SO): δ (ppm) 139.7 (q, ²J_{C-F}=37.2 Hz, C-3/C-5), 136.0 (q, ²J_{C-F}=39.6 Hz, C-3/C-5), 120.4 (q, ¹J_{C-F}=267.5 Hz, CF₃), 119.5 (q, ¹J_{C-F}=268.7 Hz, CF₃), 104.9 (C-4). IR (cm⁻¹): 2542, 2383, 1556, 1503, 1471, 1387, 1259, 1152, 1110, 1049, 1038, 878, 833, 803, 799. Raman (cm⁻¹): 2705, 2540, 1760, 1699, 1660, 1553, 1476, 1386, 1258, 1191, 997.

[(CF₃)₂Bp]Cu(C₃H₆): (FIG. 27) [(CF₃)₂Bp]Cu(NCMe) (0.15 g, 0.29 mmol) was dissolved in 5 mL dichloromethane and stirred for ˜3-5 min while bubbling propylene. The reaction mixture was concentrated with continuous flow of propylene and kept at −20° C. to obtain X-ray quality colorless crystals of [(CF₃)₂Bp]Cu(C₃H₆). Yield: >90%. M.P.: 109-112° C. (decomposition). ¹H NMR (CDCl₃): δ (ppm) 6.89 (s, 2H, PzH), 5.69-5.60 (m, 1H, ═CH), 4.73-4.69 (m, 2H, ═CH₂), 3.91 (br, 2H, BH₂), 1.74 (d, J=6.3 Hz, 3H, CH₃). ¹⁹F NMR (CDCl₃): δ (ppm) −59.8 (s), −61.2 (s). ¹³C{¹H} NMR (CDCl₃): δ (ppm) 142.0 (q, 2J_{C-F}=38.4 Hz, C-3/C-5), 139.6 (q, ²J_{C-F}=42.0 Hz, C-3/C-5), 120.2 (q, ¹J_{C-F}=268.7 Hz, CF₃), 119.2 (q, ¹J_{C-F}=271.1 Hz, CF₃), 116.0 (br, ═CH), 106.2 (C-4), 97.4 (br, =CH₂), 19.6 (CH₃). Raman (cm⁻¹), selected peak: 1543 (C═C). Structure was also confirmed by single crystal X-ray crystallography. [(CF₃)₂Bp]Cu(C₃H₆) can also be synthesized from [(CF₃)₂Bp]Cu(C₂H₄) by bubbling propylene into a dichloromethane solution of [(CF₃)₂Bp]Cu(C₂H₄). {[(CF₃)₂Bp]Cu}₃ was produced by removing propylene from [(CF₃)₂Bp]Cu(C₃H₆).

All manipulations were carried out under an atmosphere of purified nitrogen using standard Schlenk techniques or in a MBraun glovebox equipped with a −25° C. refrigerator. Solvents were purchased from commercial sources, purified before use. NMR spectra were recorded at 25° C. on a JEOL Eclipse 500 spectrometer (¹H, 500.16 MHz ¹³C, 125.78 MHz, and ¹⁹F, 470.62 MHz) unless otherwise noted. ¹H and ¹³C NMR spectra are referenced to the solvent peak (¹H; CDCl₃ δ 7.26, CD₂Cl₂ δ 5.32, (CD₃)₂SO δ 2.5, ¹³C; CDCl₃ δ 77.16, (CD₃)₂SO δ 39.52). ¹H NMR coupling constants (J) are reported in Hertz (Hz) and multiplicities are indicated as follows: s (singlet), d (doublet), t (triplet), m (multiplet). ¹⁹F NMR values were referenced to external CFCl₃. Melting points were obtained on a Mel-Temp II apparatus and were not corrected. Elemental analyses were performed using a Perkin-Elmer Model 2400 CHN analyzer. IR spectra were collected at room temperature on a Shimadzu IR Prestige-21 FTIR containing an ATR attachment using pure liquid or solid materials, with instrument resolution at 2 cm⁻¹. Raman data were collected on a Horiba Jobin Yvon LabRAM Aramin Raman spectrometer with a HeNe laser source of 633 nm, by placing pure solid materials on a glass slide. All other reactants and reagents were purchased from commercial sources. Heating was accomplished by either a heating mantle or a silicone oil bath. [(CF₃)₂Bp]Cu(NCMe)₁ was prepared via reported methods.

TABLE 1
Selected NMR and vibrational spectroscopic data of
[(CF₃)₂Bp]Cu(C₂H₄) and somewhat related ethylene complexes.

| | | Raman/IR | | | |
|---|---|---|---|---|---|
| | | (cm⁻¹) | ¹H NMR | ¹³C{¹H} NMR | |
| | Compound | (C═C) | (ppm) (C₂H₄) | (ppm) (C=C) | Reference |
| 1 | [(CF₃)₂Bp]Cu(C₂H₄) (3) | 1539 | 4.69 | 86.6 | This work |
| 2 | [(CF₃)₂Tp]Cu(C₂H₄) (2) | — | 4.96 | 89.5^{a} | Dias et al., |
| | | | | | 2002 |
| 3 | [(CF₃)₂Tp]Ag(C₂H₄) | 1573 | 5.52 | 104.9 | Dias et al., |
| | | | | | 1997; |
| | | | | | Jayratna et |
| | | | | | al., 2013 |
| 4 | [(CH₃)₂Tp]Cu(C₂H₄) (1) | — | 4.41 | — | Harlow et |
| | | | | | al., 1983 |
| 5 | [(3,5-(CF₃)₂Pz)Cu(C₂H₄)]₂ | 1537 | 4.48 (−60° C.) | 83.2 (−60° C.) | Parasar et |
| | | | | | al., 2020 |
| 6 | Free C₂H₄ | 1623 | 5.40 | 123.1 | Fulmer et |
| | | | | | al., 2010; |
| | | | | | Maslowsky |
| | | | | | et al., 2019 |

Certain NMR's were performed in solvents other than CDCl₃: ^{a}= C₆D₁₂.

X-ray Data Collection and Structure Determinations. A suitable crystal covered with a layer of hydrocarbon/Paratone-N oil was selected and mounted on a Cryo-loop, and immediately placed in the low-temperature nitrogen stream. The X-ray intensity data were measured at 100(2) K on a Bruker D8 Quest with a Photon 100 CMOS detector equipped with an Oxford Cryosystems 700 series cooler, a graphite monochromator, and a Mo Kα fine-focus sealed tube (λ=0.71073 Å). Intensity data were processed using the Bruker Apex program suite. Absorption corrections were applied by using SADABS (Krause et al., 2015. Initial atomic positions were located by SHELXT (Sheldrick, 2015-a), and the structures of the compounds were refined by the least-squares method using SHELXL (Sheldrick, 2015-b) within Olex2 GUI (Dolomanov et al., 2009). All the non-hydrogen atoms were refined anisotropically. The hydrogen atoms of ethylene and BH₂ moieties of [(CF₃)₂Bp]Cu(C₂H₄) and {[(CF₃)₂Bp]Cu}₃ were located in difference Fourier maps, included and refined freely with isotropic displacement parameters. The remaining hydrogen atoms were included in their calculated positions and refined as riding on the atoms to which they are joined. X-ray structural figures were generated using Olex2 (Dolomanov et al., 2009). CCDC 2088726-2088727 files contain the supplementary crystallographic data. These data can be obtained free of charge from the Cambridge Crystallographic Data Centre (CCDC), 12 Union Road, Cambridge, CB2 1EZ, UK).

The {[(CF₃)₂Bp]Cu}₃ crystallizes with disordered lattice solvent molecules, n-hexane. The presence of a solvent molecule could easily be seen by the residual peaks, consistent with a carbon chain, located in the open channels. Unfortunately, it was disordered over multiple positions, and also lies on a symmetry element, and therefore could not be modeled satisfactorily even with restraints. Consequently, it was removed from the electron density map using Olex2 Solvent Mask command. Based on this solvent mask calculation, and 188 electrons were found in a volume of 800 A³ in 2 voids per unit cell. This is consistent with the presence of 0.5[C₆H₁₄] per Asymmetric Unit which accounts for 200 electrons per unit cell.

TABLE 2
Crystal data and structure refinement for [(CF₃)₂Bp]Cu(C₂H₄) (3).

| Identification code | rad752 |
|---|---|
| Empirical formula | C₁₂H₈BCuF₁₂N₄ |
| Formula weight | 510.57 |
| Temperature/K | 99.99 |
| Crystal system | triclinic |
| Space group | P-1 |
| a/Å | 8.3379(4) |
| b/Å | 9.2120(5) |
| c/Å | 12.6827(7) |
| α/° | 72.051(2) |
| β/° | 73.378(2) |
| γ/° | 79.462(2) |
| Volume/Å³ | 883.20(8) |
| Z | 2 |
| ρ_{calc}g/cm³ | 1.920 |
| μ/mm⁻¹ | 1.365 |
| F(000) | 500.0 |
| Crystal size/mm³ | 0.45 × 0.36 × 0.09 |
| Radiation | Mo Kα (λ = 0.71073) |
| 2Θ range for data collection/° | 5.012 to 66.282 |
| Index ranges | −12 ≤ h ≤ 12, −14 ≤ |
| | k ≤ 14, −19 ≤ l ≤ 19 |
| Reflections collected | 14073 |
| Independent reflections | 6682 [Rᵢₙₜ = 0.0223, R_{sigma} = 0.0321] |
| Data/restraints/parameters | 6682/0/296 |
| Goodness-of-fit on F² | 1.056 |
| Final R indexes [I >= 2σ (I)] | R₁ = 0.0366, wR₂ = 0.0967 |
| Final R indexes [all data] | R₁ = 0.0425, wR₂ = 0.1030 |
| Largest diff. peak/hole / e Å⁻³ | 1.28/−0.69 |

TABLE 3
Bond Lengths for [(CF₃)₂Bp]Cu(C₂H₄) (3).

| Atom | Atom | Length/Å | Atom | Atom | Length/Å |
|---|---|---|---|---|---|
| Cu | N2 | 1.9873(13) | N1 | C3 | 1.350(2) |
| Cu | N4 | 1.9885(12) | N1 | B | 1.573(2) |
| Cu | C11 | 2.0184(16) | N2 | C1 | 1.336(2) |
| Cu | C12 | 2.0182(16) | N3 | N4 | 1.3514(17) |
| F1 | C4 | 1.321(2) | N3 | C8 | 1.3496(19) |
| F2 | C4 | 1.334(2) | N3 | B | 1.575(2) |
| F3 | C4 | 1.336(2) | N4 | C6 | 1.3380(19) |
| F4 | C5 | 1.328(2) | C1 | C2 | 1.394(2) |
| F5 | C5 | 1.343(2) | C1 | C4 | 1.491(2) |
| F6 | C5 | 1.337(2) | C2 | C3 | 1.384(2) |
| F7 | C9 | 1.336(2) | C3 | C5 | 1.493(2) |
| F8 | C9 | 1.315(2) | C6 | C7 | 1.391(2) |
| F9 | C9 | 1.316(2) | C6 | C9 | 1.488(2) |
| F10 | C10 | 1.333(2) | C7 | C8 | 1.379(2) |
| F11 | C10 | 1.331(2) | C8 | C10 | 1.494(2) |
| F12 | C10 | 1.336(2) | C11 | C12 | 1.360(3) |
| N1 | N2 | 1.3529(16) | | | |

TABLE 4
Bond Angles for [(CF₃)₂Bp]Cu(C₂H₄) (3).

| Atom | Atom | Atom | Angle/° | Atom | Atom | Atom | Angle/° |
|---|---|---|---|---|---|---|---|
| N2 | Cu | N4 | 93.28(5) | F3 | C4 | C10 | 111.52(15) |
| N2 | Cu | C11 | 113.72(7) | F4 | C5 | F5 | 107.35(14) |
| N2 | Cu | C12 | 152.99(6) | F4 | C5 | F6 | 107.41(14) |
| N4 | Cu | C11 | 152.82(7) | F4 | C5 | C3 | 110.36(14) |
| N4 | Cu | C12 | 113.48(6) | F5 | C5 | C3 | 112.26(13) |
| C12 | Cu | C11 | 39.38(7) | F6 | C5 | F5 | 106.70(14) |
| N2 | N1 | B | 117.84(12) | F6 | C5 | C3 | 112.48(14) |
| C3 | N1 | N2 | 108.97(13) | N4 | C6 | C7 | 111.09(14) |
| C3 | N1 | B | 133.18(13) | N4 | C6 | C9 | 120.57(13) |
| N1 | N2 | Cu | 115.86(10) | C7 | C6 | C9 | 128.34(14) |
| C1 | N2 | Cu | 137.16(10) | C8 | C7 | C6 | 103.60(13) |
| C1 | N2 | N1 | 106.88(12) | N3 | C8 | C7 | 109.55(13) |
| N4 | N3 | B | 117.92(12) | N3 | C8 | C10 | 123.05(14) |
| C8 | N3 | N4 | 108.95(12) | C7 | C8 | C10 | 127.28(14) |
| C8 | N3 | B | 133.13(13) | F7 | C9 | C6 | 112.32(14) |
| N3 | N4 | Cu | 115.70(9) | F8 | C9 | F7 | 104.82(16) |
| C6 | N4 | Cu | 136.83(10) | F8 | C9 | F9 | 109.46(17) |
| C6 | N4 | N3 | 106.80(12) | F8 | C9 | C6 | 112.68(14) |
| N2 | C1 | C2 | 111.21(13) | F9 | C9 | F7 | 106.50(16) |
| N2 | C1 | C4 | 120.04(13) | F9 | C9 | C6 | 110.72(14) |
| C2 | C1 | C4 | 128.73(15) | F10 | C10 | F12 | 107.69(16) |
| C3 | C2 | C1 | 103.41(13) | F10 | C10 | C8 | 113.05(14) |
| N1 | C3 | C2 | 109.52(13) | F11 | C10 | F10 | 106.90(15) |
| N1 | C3 | C5 | 123.00(14) | F11 | C10 | F12 | 107.02(14) |
| C2 | C3 | C5 | 127.47(14) | F11 | C10 | C8 | 112.23(14) |
| F1 | C4 | F2 | 106.91(16) | F12 | C10 | C8 | 109.66(15) |
| F1 | C4 | F3 | 108.65(18) | C12 | C11 | Cu | 70.30(10) |
| F1 | C4 | C1 | 111.19(14) | C11 | C12 | Cu | 70.32(10) |
| F2 | C4 | F3 | 105.83(15) | N1 | B | N3 | 106.40(12) |
| F2 | C4 | C1 | 112.46(15) | | | | |

TABLE 5
Crystal data and structure refinement for {[(CF₃)₂Bp]Cu}₃ (4).

| Identification code | rad343_0m |
|---|---|
| Empirical formula | C₃₀H₁₂B₃Cu₃F₃₆N₁₂ |
| Formula weight | 1447.57 |
| Temperature/K | 100.01 |
| Crystal system | monoclinic |
| Space group | C2/c |
| a/Å | 13.4202(7) |
| b/Å | 23.0974(12) |
| c/Å | 17.5898(9) |
| α/° | 90 |
| β/° | 102.6680(10) |
| γ/° | 90 |
| Volume/Å³ | 5319.6(5) |
| Z | 4 |
| ρ_{calc}g/cm³ | 1.807 |
| μ/mm⁻¹ | 1.354 |
| F(000) | 2808.0 |
| Crystal size/mm³ | 0.125 × 0.12 × 0.06 |
| Radiation | Mo Kα (λ = 0.71073) |
| 2Θ range for data collection/° | 5.914 to 56.562 |
| Index ranges | −17 ≤ h ≤ 17, −30 ≤ |
| | k ≤ 30, −23 ≤ l ≤ 23 |
| Reflections collected | 31852 |
| Independent reflections | 6586 [Rᵢₙₜ = 0.0250, R_{sigma} = 0.0192] |
| Data/restraints/parameters | 6586/0/392 |
| Goodness-of-fit on F² | 1.031 |
| Final R indexes [I >= 2σ (I)] | R₁ = 0.0347, wR₂ = 0.0878 |
| Final R indexes [all data] | R₁ = 0.0419, wR₂ = 0.0916 |
| Largest diff. peak/hole / e Å⁻³ | 0.96/−0.68 |

TABLE 6
Bond Lengths for {[(CF₃)₂Bp]Cu}₃ (4).

| Atom | Atom | Length/Å | Atom | Atom | Length/Å |
|---|---|---|---|---|---|
| Cul | N2 | 1.8900(17) | N1 | C3 | 1.352(3) |
| Cul | N2¹ | 1.8900(17) | N1 | B1 | 1.559(3) |
| Cu2 | N4 | 1.8955(17) | N2 | C1 | 1.332(3 |
| Cu2 | N6 | 1.8942(17) | N3 | N4 | 1.356(2) |
| F1 | C4 | 1.306(3) | N3 | C8 | 1.351(3) |
| F2 | C4 | 1.322(3) | N3 | B1 | 1.558(3) |
| F3 | C4 | 1.305(3) | N4 | C6 | 1.339(3) |
| F4 | C5 | 1.328(3) | N5 | N6 | 1.354(2) |
| F5 | C5 | 1.332(3) | N5 | C13 | 1.349(3) |
| F6 | C5 | 1.342(3) | N5 | B2 | 1.563(2) |
| F7 | C9 | 1.308(3) | N6 | C11 | 1.335(3) |
| F8 | C9 | 1.325(3) | C1 | C2 | 1.383(3) |
| F9 | C9 | 1.305(3) | C1 | C4 | 1.489(3) |
| F10 | C10 | 1.329(3) | C2 | C3 | 1.374(3) |
| F11 | C10 | 1.339(3) | C3 | C5 | 1.491(3) |
| F12 | C10 | 1.336(3) | C6 | C7 | 1.380(3) |
| F13 | C14 | 1.337(3) | C6 | C9 | 1.493(3) |
| F14 | C14 | 1.323(3) | C7 | C8 | 1.379(3) |
| F15 | C14 | 1.333(3) | C8 | C10 | 1.491(3) |
| F16 | C15 | 1.331(3) | C11 | C12 | 1.384(3) |
| F17 | C15 | 1.330(3) | C11 | C14 | 1.489(3) |
| F18 | C15 | 1.330(3) | C12 | C13 | 1.378(3) |
| N1 | N2 | 1.355(2) | C13 | C15 | 1.494(3) |

TABLE 7
Bond Angles for {[(CF₃)₂Bp]Cu}₃ (4).

| Atom | Atom | Atom | Angle/° | Atom | Atom | Atom | Angle/° |
|---|---|---|---|---|---|---|---|
| N2 | Cu1 | N2¹ | 176.32(10) | N4 | C6 | C9 | 120.90(19) |
| N6 | Cu2 | N4 | 177.46(7) | C7 | C6 | C9 | 127.62(19) |
| N2 | N1 | B1 | 118.43(16) | C8 | C7 | C6 | 103.62(18) |
| C3 | N1 | N2 | 108.45(16) | N3 | C8 | C7 | 109.71(18) |
| C3 | N1 | B1 | 133.06(17) | N3 | C8 | C10 | 123.26(19) |
| N1 | N2 | Cu1 | 119.08(13) | C7 | C8 | C10 | 126.98(19) |
| C1 | N2 | Cu1 | 133.70(14) | F7 | C9 | F8 | 105.7(2) |
| C1 | N2 | N1 | 106.84(16) | F7 | C9 | C6 | 112.91(19) |
| N4 | N3 | B1 | 118.25(16) | F8 | C9 | C6 | 112.0(2) |
| C8 | N3 | N4 | 108.57(16) | F9 | C9 | F7 | 110.1(2) |
| C8 | N3 | B1 | 133.15(17) | F9 | C9 | F8 | 104.9(2) |
| N3 | N4 | Cu2 | 117.96(12) | F9 | C9 | C6 | 110.8(2) |
| C6 | N4 | Cu2 | 135.21(14) | F10 | C10 | F11 | 106.19(19) |
| C6 | N4 | N3 | 106.61(16) | F10 | C10 | F12 | 107.72(19) |
| N6 | N5 | B2 | 118.52(14) | F10 | C10 | C8 | 113.21(19) |
| C13 | N5 | N6 | 108.60(16) | F11 | C10 | C8 | 112.68(19) |
| C13 | N5 | B2 | 132.71(16) | F12 | C10 | F11 | 106.75(18) |
| N5 | N6 | Cu2 | 118.71(13) | F12 | C10 | C8 | 109.96(18) |
| C11 | N6 | Cu2 | 134.39(14) | N6 | C11 | C12 | 111.31(18) |
| C11 | N6 | N5 | 106.81(16) | N6 | C11 | C14 | 120.56(18) |
| N2 | C1 | C2 | 111.31(18) | C12 | C11 | C14 | 128.11(19) |
| N2 | C1 | C4 | 120.28(19) | C13 | C12 | C11 | 103.61(18) |
| C2 | C1 | C4 | 128.4(2) | N5 | C13 | C12 | 109.67(18) |
| C3 | C2 | C1 | 103.77(18) | N5 | C13 | C15 | 123.44(19) |
| N1 | C3 | C2 | 109.62(18) | C12 | C13 | C15 | 126.87(19) |
| N1 | C3 | C5 | 123.09(19) | F13 | C14 | C11 | 111.61(19) |
| C2 | C3 | C5 | 127.27(19) | F14 | C14 | F13 | 106.58(19) |
| F1 | C4 | F2 | 105.8(2) | F14 | C14 | F15 | 108.69(19) |
| F1 | C4 | C1 | 112.3(2) | F14 | C14 | C11 | 112.73(19) |
| F2 | C4 | C1 | 110.6(2) | F15 | C14 | F13 | 106.84(19) |
| F3 | C4 | F1 | 106.6(2) | F15 | C14 | C11 | 110.14(19) |
| F3 | C4 | F2 | 108.2(2) | F16 | C15 | C13 | 112.6(2) |
| F3 | C4 | C1 | 112.89(19) | F17 | C15 | F16 | 107.3(2) |
| F4 | C5 | F5 | 108.1(2) | F17 | C15 | F18 | 108.0(2) |
| F4 | C5 | F6 | 106.6(2) | F17 | C15 | C13 | 109.49(19) |
| F4 | C5 | C3 | 112.94(19) | F18 | C15 | F16 | 106.2(2) |
| F5 | C5 | F6 | 106.50(18) | F18 | C15 | C13 | 113.0(2) |
| F5 | C5 | C3 | 109.86(19) | N3 | B1 | N1 | 109.87(16) |
| F6 | C5 | C3 | 112.57(19) | N5¹ | B2 | N5 | 109.5(2) |
| N4 | C6 | C7 | 111.48(18) | | | | |

¹1 − X, +Y, 1/2 − Z

### Example 3: Adsorption Isotherm and Breakthrough Experiments

Single-component gas sorption measurement. The gas sorption isotherms were collected on an automatic volumetric adsorption apparatus (Micromeritics ASAP 2020 surface area analyzer). Before the sorption measurements, the as-synthesized sample was dried under high vacuum for 12 h at 313 K, giving the activated {[(CF₃)₂Bp]Cu}₃ (4) for gas sorption analyses.

Fitting of pure-component isotherms. The pure-component isotherm data for C₂H₄ and C₂H₆ in {[(CF₃)₂Bp]Cu}₃ (4) was fitted with the single-site Langmuir-Freundlich model.

$$N = {N}_{max} \times \frac{{bp}^{1 / n}}{\begin{matrix}1 + b & {p}^{1 / n}\end{matrix}}$$

where p (unit: kPa) is the pressure of the bulk gas at equilibrium with the adsorbed phase, N (unit: mmol g⁻¹) is the adsorbed amount per mass of adsorbent, Nₘₐₓ (unit: mmol g⁻¹) is the saturation capacities, b (unit: 1/kPa) is the affinity coefficient and n represents the deviation from an ideal homogeneous surface. The fitted parameter values are presented in Table 8.

IAST calculations of adsorption selectivities. The adsorption selectivity for C₂H₄/C₂H₆ separation is defined by

$${S}_{ads} = \frac{{q}_{1} / {q}_{2}}{{p}_{1} / {p}_{2}}$$

q₁ and q₂ are the molar loadings in the adsorbed phase in equilibrium with the bulk gas phase with partial pressures p₁ and p₂.

TABLE 8
Langmuir-Freundlich parameter fits for C₂H₄ and
C₂H₆ at 298 K in {[(CF₃)₂Bp]Cu}₃ (4).

| Adsorbates | N^{max} (mmol g⁻¹) | b (kPa⁻¹) | 1/n |
|---|---|---|---|
| C₂H₄ | 1.72573 | 527.4504 | 4.23533 |
| C₂H₆ | 0.09069 | 2.23329E−4 | 1.70872 |

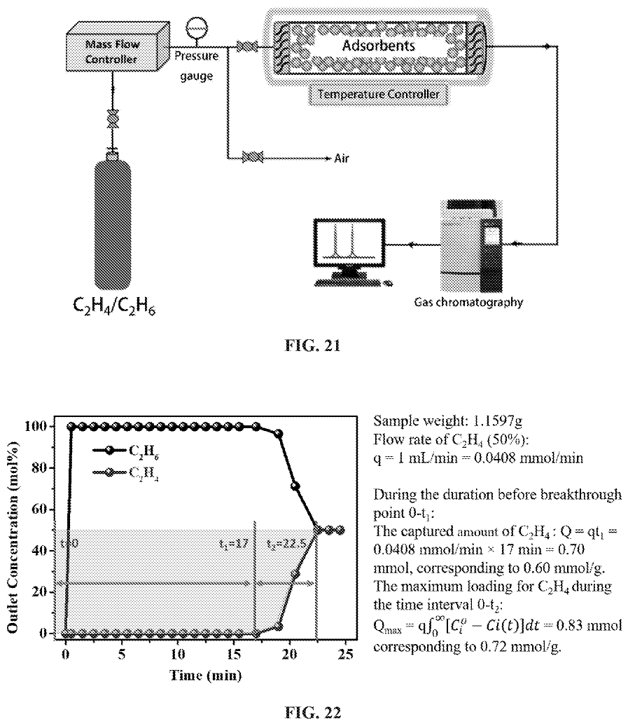

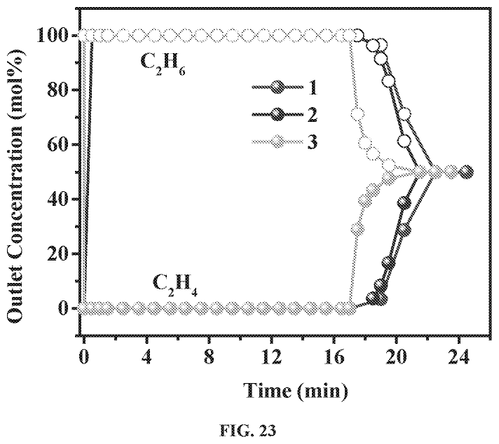

Breakthrough separation experiments. The breakthrough experiments were carried out in dynamic gas breakthrough set-up. A stainless-steel column with inner dimensions of 4×81 mm was used for sample packing. Microcrystalline sample (1.1597 g) was then packed into the column. The column was placed in a temperature-controlled environment (maintained at 298 K). The mixed gas flow and pressure were controlled by using a pressure control valve and a mass flow controller (FIG. 21). Outlet effluent from the column was continuously monitored using gas chromatography (GC-2014, SHIMADZU) with a thermal conductivity detector (TCD). The column packed with sample was firstly purged with He flow (40 mL min⁻¹) for 2 h at room temperature 298 K. The mixed gas flow rate during breakthrough process is 2 mL min⁻¹ using 50/50 (v/v) C₂H₄/C₂H₆. After the breakthrough experiment, the sample was regenerated under vacuum at 40° C. for 24 h.

In situ synchrotron powder diffraction data collection (XRD). In situ powder diffraction data of copper complexes in ethylene and He atmosphere were collected using the monochromatic X-rays available at the 17-BM. A beam (300 μm diameter beam size) with 0.45238 Å wavelength was used at the Advanced Photon Source, Argonne National Laboratory in combination with a VAREX 4343 amorphous-Si flat panel detector. A sample of analytically pure [(CF₃)₂Bp]Cu(C₂H₄) was loaded into 1.0 mm quartz capillaries with glass wool on either side. The capillary with sample was then loaded into the gas flow-cell (Chupas et al., 2008), to perform in situ PXRD experiments. At one end the gas cell was connected to a two-way valve which allowed changing between a 1 atm helium flow and a high-pressure syringe pump (Teledyne ISCO 500D) which was filled with ethene gas.

Data Processing. The raw images were processed within GSAS-II (Toby et al., 2013), refining the sample-to-detector distance and tilt of the detector relative to the beam based on data obtained for a LaB₆ standard (Hammersley et al., 1996) Collected and integrated in situ powder diffraction data sets were trimmed, normalized and plotted using 2DFLT software (Yakovenko et al., 2014).

Ethylene desorption experiment. The ethylene desorption experiment started with the pure [(CF₃)₂Bp]Cu(C₂H₄) sample in the capillary. It was heated under helium flow as ethylene desorption was allow to proceed, while monitoring composition of the system by PXRD. At around 320K, significant ethylene loss (peak position and intensity change) was started and completed at around 343K. This observation is consistent with data from NMR experiments and the synthesis of ethylene free {[(CF₃)₂Bp]Cu}₃ noted in the manuscript via separate routes.

Ethylene loading experiment. After several PXRD scans of in-situ generated sorbent, the gas flow cell was connected to the syringe pump and helium gas in the capillary was exchanged with an ethylene flow of 1 bar (100 kPa). There was peak position and intensity change immediately after application of ethylene pressure to the ethylene free sorbent, indicating formation of new crystallographic phase which was same as the [(CF₃)₂Bp]Cu(C₂H₄).

All of the compounds, material, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

### REFERENCES

The following references, to the extent that they provide exemplary procedural or other details supplementary to those set forth herein, are specifically incorporated herein by reference.
Aguado et al., J. Am. Chem. Soc., 134 (36), 14635-14637, 2012.
Bao et al., Angew. Chem., Int. Ed., 57 (49), 16020-16025, 2018.
Bereciartua et al., Science, 358 (6366), 1068-1071, 2017.
Binder, J. Biol. Chem., 295 (22), 7710-7725, 2020.
Bloch et al., Science, 335 (6076), 1606-1610, 2012.
Chu et al., Nat Mater, 16 (1), 16-22, 2016.
Chupas et al., Journal of Applied Crystallography, 41(4): p. 822-824, 2008.
Cowan et al., Angew. Chem., Int. Ed., 54 (19), 5740-5743, 2015.
Dias et al., Chem. Rev., 108 (8), 3223-3238, 2008.
Dias et al., Inorg. Chem., 36, 6205-6215, 1997.
Dias et al., J. Organomet. Chem., 690 (8), 1913-1922, 2005.
Dias et al., Organometallics, 21 (7), 1466-1473, 2002.
Dolomanov et al., J. Appl. Crystallogr., 42, 339-341, 2009.
Faiz et al., Chem. Eng. Sci., 73, 261-284, 2012.
Fulmer et al., Organometallics, 29, 2176-2179, 2010.
Geier et al., Chem. Sci., 4 (5), 2054-2061, 2013.
Groom et al., Acta Crystallogr., Sect. B Struct. Sci., Cryst. Eng. Mater., 72 (2), 171-179, 2016.
Hammersley et al., International Journal of High Pressure Research, 14(4-6): p. 235-248, 1996.
Harlow et al., J. Am. Chem. Soc., 105, 3522-3527, 1983.
He et al., Energy Environ. Sci., 5 (10), 9107-9120, 2012.
International Patent Publication No. WO 2019/183635
Jayaratna et al., Angew. Chem., Int. Ed., 57 (50), 16442-16446, 2018.
Jayaratna et al., Inorg. Chem., 52, 1691-1693, 2013.
Krause, et al., J. Appl. Crystallogr., 48, 3-10, 2015.
Li et al., Chem. Commun., 50 (86), 13081-13084, 2014.
Li et al., EnergyChem, 1 (1), 100006, 2019.
Li et al., J. Am. Chem. Soc., 136 (24), 8654-8660, 2014.
Li et al., Science, 362 (6413), 443-446, 2018.
Lin et al., Chem, 6 (2), 337-363, 2020.
Lin et al.,

Nat. Mater., 17 (12), 1128-1133, 2018.
Maslowsky, Vibrational Spectra of Organometallics: Theoretical and Experimental Data. Wiley: Hoboken, NJ 07030, USA, p 736 pp, 2019.
Mezei et al., Chem. Rev., 107 (11), 4933-5003, 2007.
Munakata et al., Inorg. Chem., 25 (15), 2622-7, 1986.
Parasar et al., Angew. Chem., Int. Ed., 59 (47), 21001-21006, 2020.
Rodriguez et al., Science, 283 (5404), 996-998, 1999.
Schaller et al., Science, 270 (5243), 1809-1811, 1995.
Schott-Verdugo et al., Sci. Rep., 9 (1), 8869, 2019.
Sheldrick et al., Acta Crystallogr. Sect. A: Found. Adv., 71, 3-8, 2015-a.
Sheldrick et al., Acta Crystallogr. Sect. C: Struct. Chem., 71, 3-8, 2015-b.
Sholl David and Lively Ryan, Nature, 532 (7600), 435-7, 2016.
Straub et al., Chem. Commun., (24), 2507-2508, 1999.
Thompson et al., J. Am. Chem. Soc., 105 (11), 3522-7, 1983.
Toby et al., Journal of Applied Crystallography, 2013, 46(2): p. 544-549, 2013.
Trofimenko, Chem. Rev., 93 (3), 943-80, 1993.
Yakovenko, 2DFLT ver.3.4.3. Upton, NY, USA: Brookhaven National Laboratory, 2014.
Yang et al., Nat. Chem., 7 (2), 121-129, 2015.
Zhang et al., Advanced Science, 7 (2), 1901918, 2020.

What is claimed is:

1. A compound of the formula: 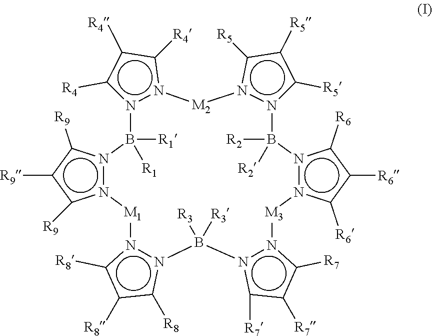
wherein:
R₁, R₁′, R₂, R₂′, R₃, and R₃′ are each independently selected from hydrogen, alkyl_{(C≤6)}, or substituted alkyl_{(C≤6)};
M₁, M₂, or M₃ are each independently selected from a monovalent metal ion; and
R₄, R₄′, R₅, R₅′, R₆, R₆′, R₇, R₇′, R₈, R₈′, R₉, and R₉′ are each independently alkyl_{(C≤12)}, substituted alkyl_{(C≤12)}, aryl_{(C≤12)}, or substituted aryl_{(C≤12)}; and
R₄″, R₅″, R₆″, R₇″, R₈″, and R₉″ is hydrogen, amino, cyano, halo, hydroxy, nitro, alkyl_{(C≤12)}, aryl_{(C≤12)}, acyl_{(C≤12)}, alkoxy_{(C≤12)}, alkylamino_{(C≤12)}, dialkylamino_{(C≤12)}, or a substituted version thereof.

2. The compound of claim 1 further defined as: 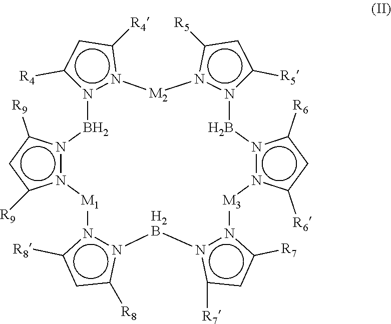
wherein:
M₁, M₂, or M₃ are each independently selected from a monovalent metal ion; and
R₄, R₄′, R₅, R₅′, R₆, R₆′, R₇, R₇′, R₈, R₈′, R₉, and R₉′ are each independently alkyl_{(C≤12)}, substituted alkyl_{(C≤12)}, aryl_{(C≤12)}, or substituted aryl_{(C≤12)}.

3. The compound of either claim 1 or claim 2 further defined as: 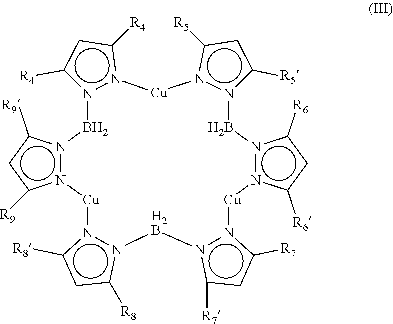
wherein:
R₄, R₄′, R₅, R₅′, R₆, R₆′, R₇, R₇′, R₈, R₈′, R₉, and R₉′ are each independently alkyl_{(C≤12)}, substituted alkyl_{(C≤12)}, aryl_{(C≤12)}, or substituted aryl_{(C≤12)}.

4. The compound according to any one of claims 1-3, wherein R₄ is substituted alkyl_{(C≤12)}.

5. The compound of claim 4, wherein R₄ is substituted alkyl_{(C≤6)}.

6. The compound of either claim 4 or claim 5, wherein R₄ is haloalkyl_{(C≤6)}.

7. The compound according to any one of claims 4-6, wherein R₄ is trifluoromethyl.

8. The compound according to any one of claims 1-7, wherein R₄′ is substituted alkyl_{(C≤12)}.

9. The compound of claim 8, wherein R₄′ is substituted alkyl_{(C≤6)}.

10. The compound of either claim 8 or claim 9, wherein R₄′ is haloalkyl_{(C≤6)}.

11. The compound according to any one of claims 8-10, wherein R₄′ is trifluoromethyl.

12. The compound according to any one of claims 1-11, wherein R₅ is substituted alkyl_{(C≤12)}.

13. The compound of claim 12, wherein R₅ is substituted alkyl_{(C≤6)}.

14. The compound of either claim 12 or claim 13, wherein R₅ is haloalkyl_{(C≤6)}.

15. The compound according to any one of claims 12-14, wherein R₅ is trifluoromethyl.

16. The compound according to any one of claims 1-15, wherein R₅′ is substituted alkyl_{(C≤12)}.

17. The compound of claim 16, wherein R₅′ is substituted alkyl_{(C≤6)}.

18. The compound of either claim 16 or claim 17, wherein R₅′ is haloalkyl_{(C≤6)}.

19. The compound according to any one of claims 16-18, wherein R₆′ is trifluoromethyl.

20. The compound according to any one of claims 1-19, wherein R₆ is substituted alkyl_{(C≤12)}.

21. The compound of claim 20, wherein R₆ is substituted alkyl_{(C≤6)}.

22. The compound of either claim 20 or claim 21, wherein R₆ is substituted alkyl_{(C≤6)}.

23. The compound according to any one of claims 20-22, wherein R₆ is trifluoromethyl.

24. The compound according to any one of claims 1-23, wherein R₆′ is substituted alkyl_{(C≤12)}.

25. The compound of claim 24, wherein R₆′ is substituted alkyl_{(C≤6)}.

26. The compound of either claim 24 or claim 25, wherein R₆′ is substituted alkyl_{(C≤6)}.

27. The compound according to any one of claims 24-26, wherein R₆′ is trifluoromethyl.

28. The compound according to any one of claims 1-27, wherein R₇ is substituted alkyl_{(C≤12)}.

29. The compound of claim 28, wherein R₇ is substituted alkyl_{(C≤6)}.

30. The compound of either claim 28 or claim 29, wherein R₇ is substituted alkyl_{(C≤6)}.

31. The compound according to any one of claims 28-30, wherein R₇ is trifluoromethyl.

32. The compound according to any one of claims 1-31, wherein R₇′ is substituted alkyl_{(C≤12)}.

33. The compound of claim 32, wherein R₇′ is substituted alkyl_{(C≤6)}.

34. The compound of either claim 32 or claim 33, wherein R₇′ is substituted alkyl_{(C≤6)}.

35. The compound according to any one of claims 32-34, wherein R₇′ is trifluoromethyl.

36. The compound according to any one of claims 1-35, wherein R₈ is substituted alkyl_{(C≤12)}.

37. The compound of claim 36, wherein R₈ is substituted alkyl_{(C≤6)}.

38. The compound of either claim 36 or claim 37, wherein R₈ is substituted alkyl_{(C≤6)}.

39. The compound according to any one of claims 36-38, wherein R⁸ is trifluoromethyl.

40. The compound according to any one of claims 1-39, wherein R₈′ is substituted alkyl_{(C≤12)}.

41. The compound of claim 40, wherein R₈′ is substituted alkyl_{(C≤6)}.

42. The compound of either claim 40 or claim 41, wherein R₈′ is substituted alkyl_{(C≤6)}.

43. The compound according to any one of claims 40-42, wherein R₈′ is trifluoromethyl.

44. The compound according to any one of claims 1-43, wherein R₉ is substituted alkyl_{(C≤12)}.

45. The compound of claim 44, wherein R₉ is substituted alkyl_{(C≤6)}.

46. The compound of either claim 44 or claim 45, wherein R₉ is substituted alkyl_{(C≤6)}.

47. The compound according to any one of claims 44-46, wherein R₉ is trifluoromethyl.

48. The compound according to any one of claims 1-47, wherein R₉′ is substituted alkyl_{(C≤12)}.

49. The compound of claim 48, wherein R₉′ is substituted alkyl_{(C≤6)}.

50. The compound of either claim 48 or claim 49, wherein R₉′ is substituted alkyl_{(C≤6)}.

51. The compound according to any one of claims 48-50, wherein R₉′ is trifluoromethyl.

52. The compound according to any one of claims 1-51, wherein the compound is further described as:

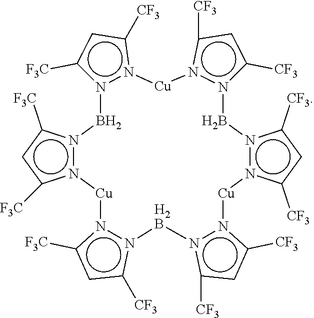

53. A compound of the formula: 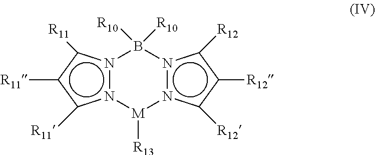
wherein:
R₁₀ and R₁₀′ are each independently selected from hydrogen, alkyl_{(C≤6)}, or substituted alkyl_{(C≤6)};
M is a monovalent metal ion; and
R₁₁, R₁₁′, R₁₂, and R₁₂′ are each independently alkyl_{(C≤12)}, substituted alkyl_{(C≤12)}, aryl_{(C≤12)}, or substituted aryl_{(C≤12)};
R₁₁″ and R₁₂″ is hydrogen, amino, cyano, halo, hydroxy, nitro, alkyl_{(C≤12)}, aryl_{(C≤12)}, acyl_{(C≤12)}, alkoxy_{(C≤12)}, alkylamino_{(C≤12)}, dialkylamino_{(C≤12)}, or a substituted version thereof; and
R₁₃ is ethylene or propylene.

54. In some embodiments, the compound further defined as: 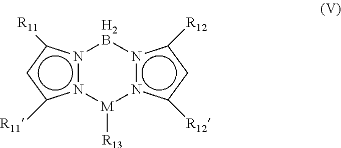
wherein:
M is a monovalent metal ion;
R₁₁, R₁₁′, R₁₂, and R₁₂′ are each independently alkyl_{(C≤12)}, substituted alkyl_{(C≤12)}, aryl_{(C≤12)}, or substituted aryl_{(C≤12)}; and
R₁₃ is ethylene or propylene.

55. The compound of either claim 53 or claim 54 further defined as: 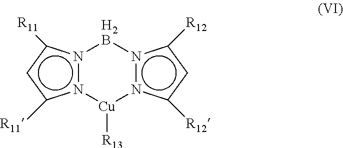
wherein:
R₁₁, R₁₁′, R₁₂, and R₁₂′ are each independently alkyl_{(C≤12)}, substituted alkyl_{(C≤12)}, aryl_{(C≤12)}, or substituted aryl_{(C≤12)}; and
R₁₃ is ethylene or propylene.

56. The compound according to any one of claims 53-55, wherein R₁₁ is substituted alkyl_{(C≤12)}.

57. The compound of claim 56, wherein R₁₁ is substituted alkyl_{(C≤6)}. 58 The compound of either claim 56 or claim 57, wherein R₁₁ is trifluoromethyl.

59. The compound according to any one of claims 53-58, wherein R₁₁′ is substituted alkyl_{(C≤12)}.

60. The compound of claim 59, wherein R₁₁′ is substituted alkyl_{(C≤6)}.

61. The compound of either claim 59 or claim 60, wherein R₁₁′ is trifluoromethyl.

62. The compound according to any one of claims 53-61, wherein R₁₂ is substituted alkyl_{(C≤12)}.

63. The compound of claim 62, wherein R₁₂ is substituted alkyl_{(C≤6)}.

64. The compound of either claim 62 or claim 63, wherein R₁₂ is trifluoromethyl.

65. The compound according to any one of claims 53-64, wherein R₁₂40 is substituted alkyl_{(C≤12)}.

66. The compound of claim 65, wherein R₁₂′ is substituted alkyl_{(C≤6)}.

67. The compound of either claim 65 or claim 66, wherein R₁₂′ is trifluoromethyl.

68. The compound according to any one of claims 53-67, wherein the compound is further defined as:

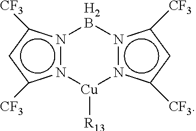

69. The compound according to any one of claims 53-68, wherein R₁₃ is ethylene.

70. The compound of claim 69, wherein R₁₃ is propylene.

71. The compound of any of claim 1-70, further defined as an organometallic complex.

72. A separation device comprising a compound according to any one of claims 1-70.

73. A method of separating an alkene_{(C≤12)} or substituted alkene_{(C≤12)} from an alkane_{(C≤12)} or substituted alkane_{(C≤12)} comprising contacting a mixture comprising an alkene_{(C≤12)} or substituted alkene_{(C≤12)} and an alkane_{(C≤12)} or substituted alkane_{(C≤12)} with a compound according to any one of claims 1-68.

74. The method of claim 73, wherein the mixture is a gas.

75. The method of either claim 73 or claim 74, wherein the method comprises a step of removing the alkane_{(C≤12)} or substituted alkane_{(C≤12)}.

76. The method of claim 75, wherein the alkane_{(C≤12)} or substituted alkane_{(C≤12)} is removed by refluxing the compound in an organic solvent.

77. The method of claim 76, wherein the organic solvent is an alkane_{(C≤8)}.

78. The method of claim 77, wherein the organic solvent is hexanes.

79. The method of claim 75, wherein the alkane_{(C≤12)} or substituted alkane_{(C≤12)} is removed under reduced pressure.

80. The method of claim 79, wherein the reduced pressure is less than 1 bar.

81. The method according to any one of claims 73, 79, and 80, wherein the temperature is from about 25° C. to about 75° C.

82. The method of claim 81, wherein the temperature is from about 30° C. to about 50° C.

83. The method according to any one of claims 73-82, wherein the mixture is contacted with the composition at a temperature from about 0° C. to about 75° C.

84. The method of claim 83, wherein the temperature is from about 20° C. to about 50° C.

85. The method of claim 84, wherein the temperature is from about 20° C. to about 30° C.

86. The method according to any one of claims 73-85, wherein the alkane_{(C≤12)} or substituted alkane_{(C≤12)} is ethane.

87. The method according to any one of claims 73-86, wherein the alkene_{(C≤12)} or substituted alkene_{(C≤12)} is ethylene.

88. The method according to any one of claims 73-85, wherein the alkane_{(C≤12)} or substituted alkane_{(C≤12)} is propane.

89. The method according to any one of claims 73-88, wherein the alkene_{(C≤12)} or substituted alkene_{(C≤12)} is propylene.

90. A method of separating propylene from propane comprising contacting a mixture comprising propylene and propane with a compound according to any one of claims 1-68.

91. A method of separating ethylene from ethane comprising contacting a mixture comprising ethylene and ethane with a compound according to any one of claims 1-68.
